(19) United States
(12) Patent Application Publication
(10) Pub. No.: US 2025/0361497 A1
(43) Pub. Date: Nov. 27, 2025

(54) MANNANASE VARIANTS AND POLYNUCLEOTIDES ENCODING SAME

(71) Applicant: Novozymes A/S, Bagsvaerd (DK)

(72) Inventors: Pengfei TIAN, Copenhagen (DK); Rolf Thomas LENHARD, Lyngby (DK); Anders Gunnar SANDSTRÖM, Sõdra Sandby (SE); Jonatan Ulrik FANGEL, Copenhagen (DK)

(73) Assignee: Novozymes A/S, Bagsvaerd (DK)

(21) Appl. No.: 18/876,373

(22) PCT Filed: Jun. 16, 2023
(86) PCT No.: PCT/EP2023/066246

§ 371 Date: Dec. 18, 2024

(30) Foreign Application Priority Data
Jun. 21, 2022 (EP) 22180103.8

(51) Int. Cl. C12N 9/24; C11D 3/386

(52) U.S. Cl. CPC C12N 9/2494; C11D 3/38636; C12Y 302/01078

(57) ABSTRACT

The present invention relates to mannanase variants with improved stability. The present invention also relates to polynucleotides encoding the variants; nucleic acid constructs, vectors, and host cells comprising the polynucleotides; and methods of using the variants.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to mannanase variants, polynucleotides encoding the variants, methods of producing the variants, and methods of using the variants. The variants of the present invention are suitable for use in cleaning processes and detergent compositions, such as laundry compositions and dishwashing compositions. In particular, the mannanase variants of the present invention show improved in-detergent stability and/or protease stability.

### Description of the Related Art

Mannans are polysaccharides with a backbone of β-1,4-linked D-mannopyranosyl residues, which can contain galactose or acetyl substitutions and may have glucose residues in the backbone. The main enzyme type participating in the degradation of mannans are endo-1,4-β-mannanases (EC 3.2.1.78), which hydrolyze the internal glycoside bonds in the mannan backbone.

Mannans are a type of hemicellulose representing up to 25% of wood dry weight in softwoods, but are also found in other plant material, especially in a variety of seeds. The mannan containing guar gum is used as a stabilizer in many food products.

Thus, it could be advantageous to use endomannanases in applications where mannan needs to be degraded. Examples of where mannanases could be used are in detergents to remove mannan containing stains, in the production of bioethanol from softwood (Vernai et al, (2011) “Synergistic action of xylanase and mannanase improves the total hydrolysis of softwood”, Bioresource tech., 102(19), pp. 9096-104) and palm kernel press cake (Jorgensen et al, (2010) “Production of ethanol and feed by high dry matter hydrolysis and fermentation of palm kernel press cake”, Applied Biochem. Biotech., 161(1-8), pp. 318-32), for the improvement of animal feed (Cai, et al, (2011), “Acidic β-mannanase from Penicillium pinophilum C1: Cloning, characterization and assessment of its potential for animal feed application”, J. Biosci. Bioeng., 112(6), pp. 551-557) and in the hydrolysis of coffee extract (Nunes et al, (2006), “Characterization of Galactomannan Derivatives in Roasted Coffee Beverages”, J. Agricultural Food Chem., 54(9), pp. 3428-3439).

Within the household care industry, it has been known to use mannanases in e.g. laundry detergents. In WO 1999/064619 an alkaline mannanase, which exhibits mannanase activity also in the alkaline pH range when applied in cleaning compositions, is disclosed.

According to CAZy (cazy.org), endo-1,4-β-mannanases have been found in glycoside hydrolyase families 5, 26 and 113. In WO 2019/068713, WO 2019/068715, WO 2021/152120 and WO 152123 mannanases of family GH 26 exhibiting beta-mannanase activity are disclosed. WO 152123 also discloses that SEQ ID NO:2 has an improved stability in the presence of protease (page 370, second last row).

### SUMMARY OF THE INVENTION

Stability under conditions relevant for the end-use, i.e. at the customer, is key for the performance and usefulness of enzymes, including mannanases. Mannanases are desirable and industrially applicable for, e.g., the detergent producing industry as discussed above. The present invention provides mannanase variants with further improved stability over the afore mentioned mannase variant having SEQ ID NO:2, in particular mannanases have improved stability, i.e. improved half life, in detergent compositions comprising protease as disclosed in Example 3.

The present invention relates to isolated mannanase variants comprising deletion at position 490 and 491 of SEQ ID NO: 2 and optionally one or more deletions or substitutions at positions corresponding to positions 16, 20, 26, 30, 36, 46, 48, 53, 61, 64, 65, 69, 70, 74, 76, 78, 82, 101, 103, 109, 111, 112, 118, 120, 126, 137, 139, 141, 143, 155, 160, 161, 162, 163, 164, 165, 166, 167, 168, 171, 172, 176, 178, 181, 182, 183, 190, 197, 214, 215, 219, 239, 244, 248, 253, 258, 271, 276, 280, 283, 286, 299, 315, 324, 366, 378, 385, 408, 410, 413, 473, 485, 486 of the polypeptide of SEQ ID NO: 2, and optionally deletion at positions corresponding to position 1, 2, 3, 4, 5, 6, 7, 8, 9, 10, 11, 12, 13, 14 and 15 of SEQ ID NO: 2, wherein the variant has at least 60% sequence identity to the polypeptide of SEQ ID NO: 3, 4, 5, 6, 7, 22, 23, 24, 25, 26, 27, 28, or 29 and wherein the variants have mannanase activity.

In a further aspect the invention concerns a variant of the polypeptide of SEQ ID NO: 2 wherein the amino acids at position 490 and 491 are deleted, wherein the variant has at least 60% sequence identity to the polypeptide of SEQ ID NO: 3 and wherein the variant has mannanase activity.

The present invention also relates to a composition comprising a variant as herein disclosed, use of such a composition in a domestic or industrial cleaning process, an isolated polynucleotide encoding the variants; nucleic acid constructs, vectors, and host cells comprising the polynucleotides; and methods of producing the variants as well as methods of washing using a composition herein disclosed.

### BRIEF DESCRIPTION OF THE FIGURES

FIG. 1 is an alignment of the polypeptides of SEQ ID NOs: 1, 2, 3, 4, 5, 6 and 7.

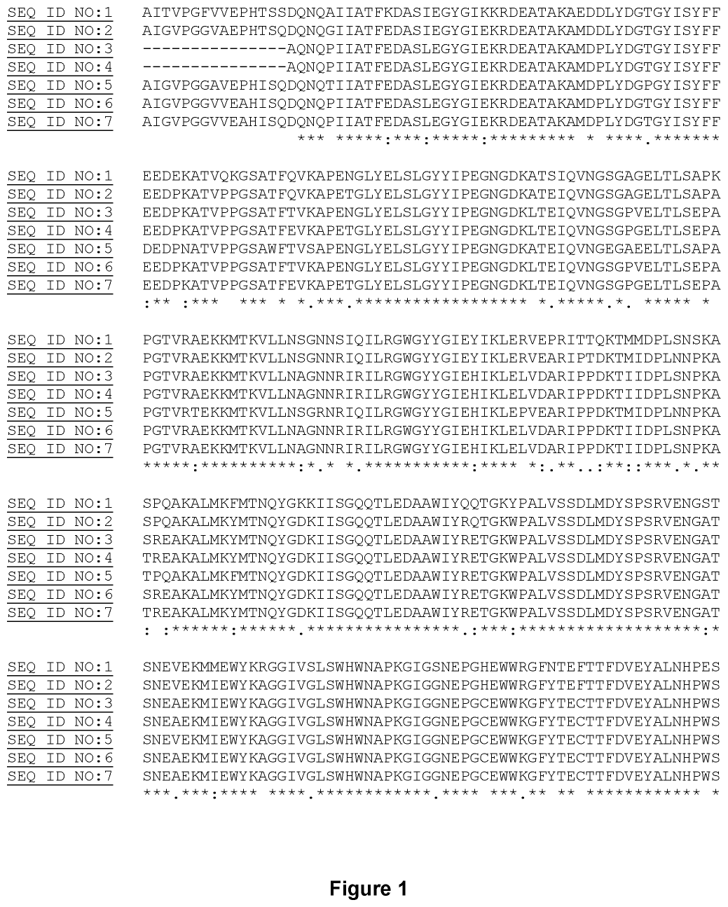

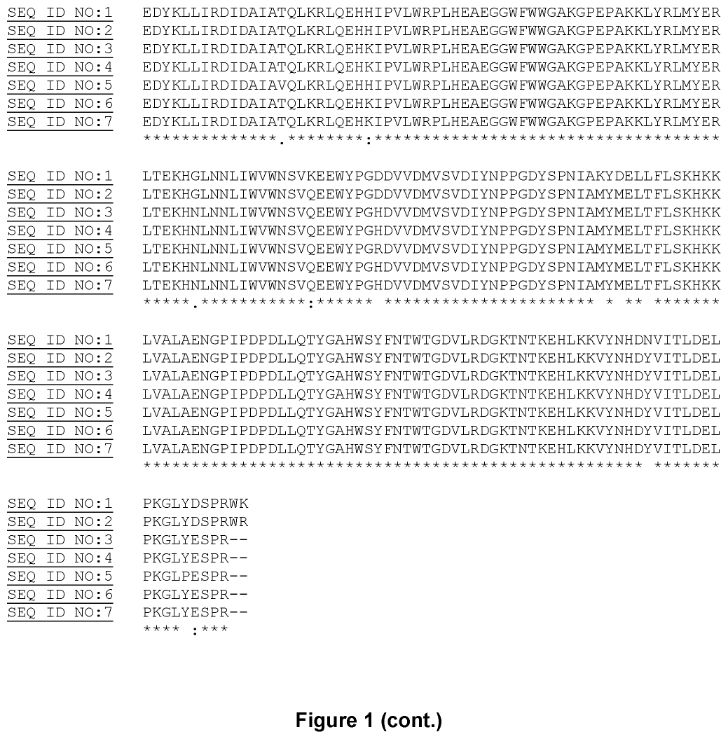

### OVERVIEW OF SEQUENCE LISTING

SEQ ID NO:1 is the mature mannanase polypeptide obtained from Paenibacillus illinoisensis

SEQ ID NO:2 is the mature mannanase polypeptide obtained from Paenibacillus illinoisensis which is a variant of SEQ ID NO:1

SEQ ID NO:3 to SEQ ID NO: 7, SEQ ID NO:9 to SEQ ID NO: 16 and SEQ ID NO:22 to

SEQ ID NO: 29 are variants of SEQ ID NO:2

SEQ ID NO:8 is a GH5 mannanase from Bacillus bogoriensis

SEQ ID NO: 17 is a protease from Bacillus lentus

SEQ ID NO: 18 is a mannanase from Bacillus sp.

SEQ ID NO: 19 is a mannanase from Bacillus clausii

SEQ ID NO: 20 is a mannanase from Bacillus lentus

SEQ ID NO: 21 is a mannanase from Paenibacillus sp.

SEQ ID NO: 30 is a xanthan lyase from a strain of a Paenibacillus sp

SEQ ID NO: 31 is a xanthan endoglucanase from a strain of a Paenibacillus sp

SEQ ID NO: 32 is a protease from Bacillus lentus

### Definitions

In accordance with this detailed description, the following definitions apply. Note that the singular forms “a,” “an,” and “the” include plural references unless the context clearly dictates otherwise.

Unless defined otherwise or clearly indicated by context, all technical and scientific terms used herein have the same meaning as commonly understood by one of ordinary skill in the art to which this invention belongs.

Mannanase Activity: For estimating the mannose yield after substrate hydrolysis, a reducing end assay developed by Lever (1972), Anal. Biochem. 47: 273-279, is used. The assay is based on 4-hydroxybenzoic acid hydrazide, which under alkaline conditions reacts with the reducing ends of saccharides. The product is a strong yellow anion, which absorbs at 410 nm.

Mannanase activity of a variant is determined as described in Example 1.

Adjunct materials: The term “adjunct materials” or “adjunct ingredients” means any liquid, solid or gaseous material selected for the particular type of detergent composition desired and the form of the product (e.g., liquid, granule, powder, bar, paste, spray, tablet, gel, or foam composition), which materials are also preferably compatible with the mannanase variant enzyme used in the composition. More detailed information on adjunct materials is provided further below.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences involved in regulation of expression of a polynucleotide in a specific organism or in vitro. Each control sequence may be native (i.e., from the same gene) or heterologous (i.e., from a different gene) to the polynucleotide encoding the variant, and native or heterologous to each other. Such control sequences include, but are not limited to leader, polyadenylation, prepropeptide, propeptide, signal peptide, promoter, terminator, enhancer, and transcription or translation initiator and terminator sequences. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Detergent composition: The term “detergent composition” (or “cleaning composition”) includes unless otherwise indicated any form of detergent or cleaning composition. These include granular or powder-form all-purpose or heavy-duty washing agents, especially cleaning detergents; liquid, gel or paste-form all-purpose washing agents, especially the so-called heavy-duty liquid (HDL) types; single unit dose (SUD) compositions such as pods, capsules, tabs, etc. with one or more chambers; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, soap bars, mouthwashes, denture cleaners, car or carpet shampoos, bathroom cleaners; hair shampoos and hair-rinses; shower gels, foam baths; metal cleaners; as well as cleaning auxiliaries such as bleach additives and “stain-stick” or pre-treat types. The terms “detergent composition” and “detergent formulation” are used in reference to mixtures which are intended for use in a wash medium for the cleaning of soiled objects. In some embodiments, the term is used in reference to laundering fabrics and/or garments (e.g., “laundry detergents”). In alternative embodiments, the term refers to other detergents, such as those used to clean dishes, cutlery, etc. (e.g., “dishwashing detergents”). It is not intended that the present invention be limited to any particular detergent formulation or composition. The term “detergent composition” is not intended to be limited to compositions that contain surfactants. It is intended that in addition to the variants according to the invention, the term encompasses detergents that may contain, e.g., surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferases, hydrolytic enzymes, oxido reductases, bluing agents and fluorescent dyes, antioxidants, and solubilizers.

Effective amount of enzyme: The term “effective amount of enzyme” refers to the quantity of enzyme necessary to achieve the enzymatic activity required in the specific application, e.g., in a defined detergent composition. Such effective amounts are readily ascertained by one of ordinary skill in the art and are based on many factors, such as the particular enzyme used, the cleaning application, the specific composition of the detergent composition, and whether a liquid or dry (e.g., granular, bar) composition is required, and the like. The term “effective amount” of a mannanase variant refers to the quantity of mannanase variant described hereinbefore that achieves a desired level of enzymatic activity, e.g., in a defined detergent composition.

Expression: The term “expression” includes any step involved in the production of a variant including, but not limited to, transcription, post-transcriptional modification, translation, post-translational modification, and secretion.

Expression vector: An “expression vector” refers to a linear or circular DNA construct comprising a DNA sequence encoding a variant, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

Fabric: The term “fabric” encompasses any textile material. Thus, it is intended that the term encompass garments, as well as fabrics, yarns, fibers, non-woven materials, natural materials, synthetic materials, and any other textile material.

Extension: The term “extension” means an addition of one or more amino acids to the amino and/or carboxyl terminus of a variant, wherein the “extended” variant has mannanase activity.

Fragment: The term “fragment” means a variant having one or more amino acids absent from the amino and/or carboxyl terminus of the variant; wherein the fragment has mannanase activity. In one aspect, the fragment comprises at least amino acids 13 to 489 of SEQ ID NO: 5, such as at least amino acids 14 to 489 of SEQ ID NO: 5, such as at least amino acids 15 to 489 of SEQ ID NO: 5. In another aspect, the fragment comprises at least amino acids 13 to 489 of SEQ ID NO: 6, such as at least amino acids 14 to 489 of SEQ ID NO: 6, such as at least amino acids 15 to 489 of SEQ ID NO: 6. In yet another aspect, the fragment comprises at least amino acids 13 to 489 of SEQ ID NO: 7, such as at least amino acids 14 to 489 of SEQ ID NO: 7, such as at least amino acids 15 to 489 of SEQ ID NO: 7.

Hard surface cleaning: The term “Hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, and cutlery such as spoons, knives, forks, serving utensils, ceramics, plastics, metals, china, glass and acrylics.

Hemicellulolytic enzyme or hemicellulase: The term “hemicellulolytic enzyme” or “hemicellulase” means one or more (e.g., several) enzymes that hydrolyze a hemicellulosic material. See, for example, Shallom and Shoham, Current Opinion In Microbiology, 2003, 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, of a polypeptide or nucleic acid is not naturally associated with the polypeptide or nucleic acid, i.e., the control sequence is from a gene other than the gene encoding the mature polypeptide.

Host Strain or Host Cell: A “host strain” or “host cell” is an organism into which an expression vector, phage, virus, or other DNA construct, including a polynucleotide encoding a variant has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

Improved property: The term “improved property” means a characteristic associated with a variant that is improved compared to the parent. Such improved properties include, but are not limited to, in-detergent stability, thermostability, protease stability, surfactant stability, pH stability.

Improved wash performance: The term “improved wash performance” may be defined as improved cleaning effect of a mannanase variant according to the invention compared to the mannanase with SEQ ID NO: 1 or SEQ ID NO: 2. Wash performance may be expressed as a remission value of the stained swatches. After washing and rinsing the swatches are spread out flat and allowed to air dry at room temperature overnight. All washed swatches are evaluated the day after washing. Light reflectance evaluations of the swatches are done using a Macbeth Color Eye 7000 reflectance spectrophotometer with very small aperture. The measurements are made without UV in the incident light and remission value at 460 nm is extracted.

In-detergent stability: The term “in-detergent stability” or “detergent stability” refers to the stability of a mannanase enzyme, whether a wild-type, parent, or variant, which has been incubated in detergent. For purposes of the present invention, in-detergent stability may be determined as shown in the Example 3.

Introduced: The term “introduced” in the context of inserting a nucleic acid sequence into a cell, means “transfection”, “transformation” or “transduction,” as known in the art.

Isolated: The term “isolated” means a variant, nucleic acid, cell, or other specified material or component that is separated from at least one other material or component, including, but not limited to, other proteins, nucleic acids, cells, etc. An isolated polypeptide, nucleic acid, cell or other material is thus in a form that does not occur in nature. An isolated polypeptide includes, but is not limited to, a culture broth containing the secreted variant expressed in a host cell.

Laundering: The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

Mannanase: The term “mannanase” means a polypeptide having mannan endo-1,4-beta-mannosidase activity (EC 3.2.1.78) that catalyzes the hydrolysis of 1,4-β-D-mannosidic linkages in mannans, galactomannans and glucomannans. Alternative names of mannan endo-1,4-beta-mannosidase are 1,4-β-D-mannan mannanohydrolase; endo-1,4-β-mannanase; endo-β-1,4-mannase; β-mannanase B; β-1,4-mannan 4-mannanohydrolase; endo-β-mannanase; and β-D-mannanase.

According to CAZy (www.cazy.org) mannanases can be found in two groups: Glycoside Hydrolase Family 5 (GH5) and Glycoside Hydrolase Family 26 (GH26). The substrate specificity varies for the two groups and a combination of GH5 and GH26 may thus be advantageous.

Mannanases belonging to GH5 family are disclosed in WO2018/206300 and WO2018/206302.

Mannanases belonging to GH26 family are disclosed in WO2019/068715, WO2021/152120 and WO152123.

For purposes of the present invention, mannanase activity may be determined using the Reducing End Assay as described in Example 1 herein.

Mature polypeptide: The term “mature polypeptide” means a polypeptide in its mature form following N-terminal processing and/or C-terminal processing (e.g., removal of signal peptide). In one aspect, the mature polypeptide is amino acids 1 to 474 of SEQ ID NO: 3. In one aspect, the mature polypeptide is amino acids 1 to 474 of SEQ ID NO: 4. In one aspect, the mature polypeptide is amino acids 1 to 489 of SEQ ID NO: 5. In one aspect, the mature polypeptide is amino acids 1 to 489 of SEQ ID NO: 6. In one aspect, the mature polypeptide is amino acids 1 to 489 of SEQ ID NO: 7.

Mature polypeptide coding sequence: The term “mature polypeptide coding sequence” means a polynucleotide that encodes a mature polypeptide having mannnase activity.

Modification: The term “modification”, in the context of the polypeptides of the invention, means that one or more amino acids within the reference amino acid sequence (i.e. SEQ ID NOs: 1 or 2) are altered by substitution with a different amino acid, by insertion of an amino acid or by deletion, preferably by at least one deletion. The terms “modification”, “alteration”, and “mutation” may be used interchangeably and constitute the same meaning and purpose.

Mutant: The term “mutant” means a polynucleotide encoding a variant.

Native: The term “native” means a nucleic acid or polypeptide naturally occurring in a host cell.

Nucleic acid: The term “nucleic acid” encompasses DNA, RNA, heteroduplexes, and synthetic molecules capable of encoding a variant. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5-to-3′ orientation.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, and which comprises one or more control sequences operably linked to the nucleic acid sequence.

Operably linked: The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequence.

Parent or parent mannanase: The term “parent” or “parent mannanase” means a mannanase to which an alteration is made to produce the enzyme variants of the present invention. The mannanase having SEQ ID NO:2 is a parent mannanase.

Protease stability: The term “protease stability” refers to the stability of a mannanase enzyme, whether a wild-type, parent, or variant, which has been incubated in in the presence of a protease, optionally in a detergent. For purposes of the present invention, protease stability may be determined as shown in the Example 4.

Purified: The term “purified” means a nucleic acid, variant or cell that is substantially free from other components as determined by analytical techniques well known in the art (e.g., a purified variant or nucleic acid may form a discrete band in an electrophoretic gel, chromatographic eluate, and/or a media subjected to density gradient centrifugation). A purified nucleic acid or variant is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight or on a molar basis). In a related sense, a composition is enriched for a molecule when there is a substantial increase in the concentration of the molecule after application of a purification or enrichment technique. The term “enriched” refers to a compound, variant, cell, nucleic acid, amino acid, or other specified material or component that is present in a composition at a relative or absolute concentration that is higher than a starting composition.

In one aspect, the term “purified” as used herein refers to the variant or cell being essentially free from components (especially insoluble components) from the production organism. In other aspects, the term “purified” refers to the variant being essentially free of insoluble components (especially insoluble components) from the native organism from which it is obtained. In one aspect, the variant is separated from some of the soluble components of the organism and culture medium from which it is recovered. The variant may be purified (i.e., separated) by one or more of the unit operations filtration, precipitation, or chromatography.

Accordingly, the variant may be purified such that only minor amounts of other proteins, in particular, other polypeptides, are present. The term “purified” as used herein may refer to removal of other components, particularly other proteins and most particularly other enzymes present in the cell of origin of the polypeptide. The variant may be “substantially pure”, i.e., free from other components from the organism in which it is produced, e.g., a host organism for recombinantly produced variant. In one aspect, the polypeptide is at least 40% pure by weight of the total polypeptide material present in the preparation. In one aspect, the polypeptide is at least 50%, 60%, 70%, 80% or 90% pure by weight of the total polypeptide material present in the preparation. As used herein. a “substantially pure polypeptide” may denote a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which the polypeptide is natively or recombinantly associated.

It is, therefore, preferred that the substantially pure variant is at least 92% pure, preferably at least 94% pure, more preferably at least 95% pure, more preferably at least 96% pure, more preferably at least 97% pure, more preferably at least 98% pure, even more preferably at least 99% pure, most preferably at least 99.5% pure by weight of the total polypeptide material present in the preparation. The variant of the present invention is preferably in a substantially pure form (i.e., the preparation is essentially free of other polypeptide material with which it is natively or recombinantly associated). This can be accomplished, for example by preparing the variant by well-known recombinant methods or by classical purification methods.

Recombinant: The term “recombinant” is used in its conventional meaning to refer to the manipulation, e.g., cutting and rejoining, of nucleic acid sequences to form constellations different from those found in nature. The term recombinant refers to a cell, nucleic acid, variant or vector that has been modified from its native state. Thus, for example, recombinant cells express genes that are not found within the native (non-recombinant) form of the cell, or express native genes at different levels or under different conditions than found in nature. The term “recombinant” is synonymous with “genetically modified” and “transgenic”.

Recover: The terms “recover” or “recovery” means the removal of a polypeptide from at least one fermentation broth component selected from the list of a cell, a nucleic acid, or other specified material, e.g., recovery of the polypeptide from the whole fermentation broth, or from the cell-free fermentation broth, by polypeptide crystal harvest, by filtration, e.g., depth filtration (by use of filter aids or packed filter medias, cloth filtration in chamber filters, rotary-drum filtration, drum filtration, rotary vacuum-drum filters, candle filters, horizontal leaf filters or similar, using sheed or pad filtration in framed or modular setups) or membrane filtration (using sheet filtration, module filtration, candle filtration, microfiltration, ultrafiltration in either cross flow, dynamic cross flow or dead end operation), or by centrifugation (using decanter centrifuges, disc stack centrifuges, hyrdo cyclones or similar), or by precipitating the polypeptide and using relevant solid-liquid separation methods to harvest the polypeptide from the broth media by use of classification separation by particle sizes. Recovery encompasses isolation and/or purification of the polypeptide.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the −nobrief option must be specified in the command line. The output of Needle labeled “longest identity” is calculated as follows:

$$( Identical Residues \times 100 ) / ( Length of Alignment - Total Number of Gaps in Alignment )$$

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

$$( Identical Deoxyribonucleotides \times 100 ) / ( Length of Alignment - Total Number of Gaps in Alignment )$$

Signal Peptide: A “signal peptide” is a sequence of amino acids attached to the N-terminal portion of a protein, which facilitates the secretion of the protein outside the cell. The mature form of an extracellular protein lacks the signal peptide, which is cleaved off during the secretion process.

Subsequence: The term “subsequence” means a polynucleotide having one or more nucleotides absent from the 5′ and/or 3′ end of a mature polypeptide coding sequence; wherein the subsequence encodes a fragment having mannanase activity.

Surfactant stability: The term “surfactant stability” refers to the stability of a mannanase enzyme, whether a wild-type, parent, or variant, which has been incubated in the presence of a surfactant, for example in the presence of a surfactant in a detergent. Exemplary surfactants are those described in detail below, and in a particular embodiment, surfactant stability refers to stability in the presence of an anionic surfactant, such as LAS. For purposes of the present invention, surfactant stability may be determined as shown in the Examples.

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, ramie, cellulose acetate fibers (tricell), lyocell or blends thereof. The textile or fabric may also be non-cellulose based such as natural polyamides including wool, camel, cashmere, mohair, rabit and silk or synthetic polymer such as nylon, aramid, polyester, acrylic, polypropylen and spandex/elastane, or blends thereof as well as blend of cellulose based and non-cellulose based fibers. Examples of blends are blends of cotton and/or rayon/viscose with one or more companion material such as wool, synthetic fibers (e.g. polyamide fibers, acrylic fibers, polyester fibers, polyvinyl alcohol fibers, polyvinyl chloride fibers, polyurethane fibers, polyurea fibers, aramid fibers), and cellulose-containing fibers (e.g. rayon/viscose, ramie, flax/linen, jute, cellulose acetate fibers, lyocell). Fabric may be conventional washable laundry, for example stained household laundry. When the term fabric or garment is used it is intended to include the broader term textiles as well.

Thermostability: The term “thermostability” refers to the stability of a mannanase enzyme, whether a wild-type, parent, or variant, which has been incubated in the presence of a elevated temperature. For purposes of the present invention, thermostability may be determined using nDSF (Prometheus, Nanotemper) by measuring melting temperature (Tm) of variants in presence of detergent (for example, Model Detergent of Table 1). Samples are prepared to achieve Model Detergent of Table 1 concentration of 10% and protein concentration of 200 ppm. Samples after brief mixing are loaded into capillaries and placed in nDSF sample tray. Samples are run in Tm estimation mode with temperature range of 25-85° C. with 1° C./min ramp. After completion of run, Tm is estimated using 350 nm signal in the software

Variant: The term “variant” means a polypeptide having mannanase activity comprising a substitution, an insertion (including extension), and/or a deletion (e.g., truncation), at one or more positions. A substitution means replacement of the amino acid occupying a position with a different amino acid; a deletion means removal of the amino acid occupying a position; and an insertion means adding 1-5 amino acids (e.g., 1-3 amino acids, in particular, 1 amino acid) adjacent to and immediately following the amino acid occupying a position.

Wash liquor: The term “wash liquor” refers to an aqueous solution comprising a mannanase variant of the invention. A wash liquor is a solution, e.g. found in a washing machine or dishwasher, containing water and a detergent composition comprising the mannanase. The detergent composition, prior to being mixed with water to form a wash liquor, may be in any form as described elsewhere herein, for example a liquid or powder.

Water hardness: The term “water hardness” or “degree of hardness” or “dH” or “° dH” as used herein refers to German degrees of hardness. One degree is defined as 10 milligrams of calcium oxide per liter of water.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

### Conventions for Designation of Variants

For purposes of the present invention, the polypeptide disclosed in SEQ ID NO: 2 is used to determine the corresponding amino acid positions in another mannanase. The amino acid sequence of another mannanase is aligned with the polypeptide disclosed in SEQ ID NO: 2, and based on the alignment, the amino acid position number corresponding to any amino acid residue in the polypeptide disclosed in SEQ ID NO: 2 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are gap open penalty of 10, gap extension penalty of 0.5, and the EBLOSUM62 (EMBOSS version of BLOSUM62) substitution matrix.

In describing the variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions. For an amino acid insertion, the following nomenclature is used: Original amino acid, position, original amino acid, inserted amino acid. Accordingly, the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different alterations. Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants: “Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

The present invention relates to isolated mannanase variants comprising deletion at position 490 and 491 of SEQ ID NO: 2 and optionally one or more deletions or substitutions at positions corresponding to positions 16, 20, 26, 30, 36, 46, 48, 53, 61, 64, 65, 69, 70, 74, 76, 78, 82, 101, 103, 109, 111, 112, 118, 120, 126, 137, 139, 141, 143, 155, 160, 161, 162, 163, 164, 165, 166, 167, 168, 171, 172, 176, 178, 181, 182, 183, 190, 197, 214, 215, 219, 239, 244, 248, 253, 258, 271, 276, 280, 283, 286, 299, 315, 324, 366, 378, 385, 408, 410, 413, 473, 485, 486 of the polypeptide of SEQ ID NO: 2, and optionally deletion at positions corresponding to position 1, 2, 3, 4, 5, 6, 7, 8, 9, 10, 11, 12, 13, 14 and 15 of SEQ ID NO: 2, wherein the variant has at least 60% sequence identity to the polypeptide of SEQ ID NO: 3, 4, 5, 6, 7, 22, 23, 24, 25, 26, 27, 28, or 29 and wherein the variants have mannanase activity.

In a further aspect the invention concerns a variant of the polypeptide of SEQ ID NO: 2 wherein the amino acids at position 490 and 491 are deleted, wherein the variant has mannanase activity, and wherein the variant has at least 60%, e.g., at 65%, 70%, 75%, 80%, 85%, 85.5%, 86%, 86.5%, 87%, 87.5%, 88%, 88.5%, 89%, 89.5%, 90%, 90.5%, at least 90.6%, at least 90.7%, at least 90.8%, at least 90.9%, such as at least 91%, at least 91.1%, at least 91.2%, at least 91.3%, at least 91.4%, at least 91.5%, at least 91.6%, at least 91.7%, at least 91.8%, at least 91.9%, such as at least 92%, at least 92.1%, at least 92.2%, at least 92.3%, at least 92.4%, at least 92.5%, at least 92.6%, at least 92.7%, at least 92.8%, at least 92.9%, such as at least 93%, at least 93.1%, at least 93.2%, at least 93.3%, at least 93.4%, at least 93.5%, at least 93.6%, at least 93.7%, at least 93.8%, at least 93.9%, such as at least 94%, at least 94.1%, at least 94.2%, at least 94.3%, at least 94.4%, at least 94.5%, at least 94.6%, at least 94.7%, at least 94.8%, at least 94.9%, such as at least 95%, at least 95.1%, at least 95.2%, at least 95.3%, at least 95.4%, at least 95.5%, at least 95.6%, at least 95.7%, at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, such as at least 97%, at least 97.1%, at least 97.2%, at least 97.3%, at least 97.4%, at least 97.5%, at least 97.6%, at least 97.7%, at least 97.8%, at least 97.9%, such as at least 98%, at least 98.1%, at least 98.2%, at least 98.3%, at least 98.4%, at least 98.5%, at least 98.6%, at least 98.7%, at least 98.8%, at least 98.9%, such as at least 99%, at least 99.1%, at least 99.2%, at least 99.3%, at least 99.4%, at least 99.5%, at least 99.6%, at least 99.7%, at least 99.8%, at least 99.9% sequence identity or even 100% sequence identity to the polypeptide of SEQ ID NO: 3, the polypeptide of SEQ ID NO: 4, the polypeptide of SEQ ID NO: 5, the polypeptide of SEQ ID NO: 6, the polypeptide of SEQ ID NO: 7, the polypeptide of SEQ ID NO: 22, the polypeptide of SEQ ID NO: 23, the polypeptide of SEQ ID NO: 24, the polypeptide of SEQ ID NO: 25, the polypeptide of SEQ ID NO: 26, the polypeptide of SEQ ID NO: 27, the polypeptide of SEQ ID NO: 28, or the polypeptide of SEQ ID NO: 29 and wherein the variants have mannanase activity. In a particular embodiment the variants comprise a truncation of one or more amino acids at the N-terminal end.

### Variants

In the following, unless otherwise explicitly stated, the numbering of amino acids of the variants refers to the numbering obtained when aligned with SEQ ID:2.

In one embodiment the mannanase variant of the present invention comprises the deletions W490* and R491* of SEQ ID NO:2.

In a certain preferred embodiment the variant in addition to the deletion of amino acids corresponding to the amino acids of position W490* and R491* comprises deletion of at least 11 but less than 16 amino acids from N-terminal end of the polypeptide having SEQ ID NO:2. The deletion may comprise 11-15, such as 11, 12, 13, 14 or 15 amino acids from the N-terminal end of the polypeptide having SEQ ID NO:2. In a certain preferred embodiment the variant comprises the deletions A1*+I2*+G3*+V4*+P5*+G6*+G7*+V8*+A9*+E10*+P11*+H12*+T13*+S14*+Q15* of SEQ ID NO:2. In particular, when 15 amino acids are deleted from the N-terminal end of the polypeptide having SEQ ID NO:2, the variant may further comprise the substitution D16A.

In a further embodiment the variant comprises in addition to the deletions and substitution disclosed in the embodiments above, one or more modifications selected from the group consisting of I30L, D48P, Y155H, T167P, Q215E, H276C, R280K, F286C, G366N, and D486E of SEQ ID NO:2.

In an embodiment the variant comprises the substitutions H276C+R280K+F286C and optionally one or more substitutions selected from I30L, D48P, Y155H, T167P, Q215E, G366N, and D486E, such as the substitutions D48P+H276C+R280K+F286C, T167P+H276C+R280K+F286C, Q215E+H276C+R280K+F286C, D48P+T167P+H276C+R280K+F286C, D48P+Q215E+H276C+R280K+F286C, T167P+Q215E+H276C+R280K+F286C or D48P+T167P+Q215E+H276C+R280K+F286C of SEQ ID NO:2.

In an embodiment the variant of SEQ ID NO:2 comprises at least two of the substitutions D48P, T167P and Q215E, such as D48P+T167P and optionally one or more substitutions selected from I30L, Y155H, H276C, R280K, F286C G366N, and D486E.

In an embodiment the variant of SEQ ID NO:2 comprises at least two of the substitutions D48P, T167P and Q215E, such as D48P+Q215E and optionally one or more substitutions selected from I30L, Y155H, H276C, R280K, F286C G366N, and D486E.

In an embodiment the variant of SEQ ID NO:2 comprises at least two of the substitutions D48P, T167P and Q215E, such as T167P+Q215E and optionally one or more substitutions selected from I30L, Y155H, H276C, R280K, F286C G366N, and D486E.

In an embodiment the variant of SEQ ID NO:2 comprises at least two of the substitutions D48P, T167P and Q215E, such as D48P+T167P+Q215E and optionally one or more substitutions selected from I30L, Y155H, H276C, R280K, F286C G366N, and D486E.

In one aspect, a variant comprises substitutions at positions corresponding to positions selected from a group consisting of: I30L, D48P, Y155H, T167P, Q215E, H276C, R280K, F286C, G366N, D486E, I30L+D48P, I30L+Y155H, I30L+T167P, I30L+Q215E, I30L+H276C, I30L+R280K, I30L+F286C, I30L+G366N, I30L+D486E, D48P+Y155H, D48P+T167P, D48P+Q215E, D48P+H276C, D48P+R280K, D48P+F286C, D48P+G366N, D48P+D486E, Y155H+T167P, Y155H+Q215E, Y155H+H276C, Y155H+R280K, Y155H+F286C, Y155H+G366N, Y155H+D486E, T167P+Q215E, T167P+H276C, T167P+R280K, T167P+F286C, T167P+G366N, T167P+D486E, Q215E+H276C, Q215E+R280K, Q215E+F286C, Q215E+G366N, Q215E+D486E, H276C+R280K, H276C+F286C, H276C+G366N, H276C+D486E, R280K+F286C, R280K+G366N, R280K+D486E, F286C+G366N, F286C+D486E, G366N+D486E, I30L+D48P+Y155H, I30L+D48P+T167P, I30L+D48P+Q215E, I30L+D48P+H276C, I30L+D48P+R280K, I30L+D48P+F286C, I30L+D48P+G366N, I30L+D48P+D486E, I30L+Y155H+T167P, I30L+Y155H+Q215E, I30L+Y155H+H276C, I30L+Y155H+R280K, I30L+Y155H+F286C, I30L+Y155H+G366N, I30L+Y155H+D486E, I30L+T167P+Q215E, I30L+T167P+H276C, I30L+T167P+R280K, I30L+T167P+F286C, I30L+T167P+G366N, I30L+T167P+D486E, 30L+Q215E+H276C, I30L+Q215E+R280K, I30L+Q215E+F286C, I30L+Q215E+G366N, I30L+Q215E+D486E, I30L+H276C+R280K, I30L+H276C+F286C, I30L+H276C+G366N, I30L+H276C+D486E, I30L+R280K+F286C, I30L+R280K+G366N, I30L+R280K+D486E, I30L+F286C+G366N, I30L+F286C+D486E, I30L+G366N+D486E, D48P+Y155H+T167P, D48P+Y155H+Q215E, D48P+Y155H+H276C, D48P+Y155H+R280K, D48P+Y155H+F286C, D48P+Y155H+G366N, D48P+Y155H+D486E, D48P+T167P+Q215E, D48P+T167P+H276C, D48P+T167P+R280K, D48P+T167P+F286C, D48P+T167P+G366N, D48P+T167P+D486E, D48P+Q215E+H276C, D48P+Q215E+R280K, D48P+Q215E+F286C, D48P+Q215E+G366N, D48P+Q215E+D486E, D48P+H276C+R280K, D48P+H276C+F286C, D48P+H276C+G366N, D48P+H276C+D486E, D48P+R280K+F286C, D48P+R280K+G366N, D48P+R280K+D486E, D48P+F286C+G366N, D48P+F286C+D486E, D48P+G366N+D486E, Y155H+T167P+Q215E, Y155H+T167P+H276C Y155H+T167P+R280K, Y155H+T167P+F286C, Y155H+T167P+G366N, Y155H+T167P+D486E, Y155H+Q215E+H276C, Y155H+Q215E+R280K, Y155H+Q215E+F286C, Y155H+Q215E+G366N, Y155H+Q215E+D486E, Y155H+H276C+R280K, Y155H+H276C+F286C, Y155H+H276C+G366N, Y155H+H276C+D486E, Y155H+R280K+F286C, Y155H+R280K+G366N, Y155H+R280K+D486E, Y155H+F286C+G366N, Y155H+F286C+D486E, Y155H+G366N+D486E, T167P+Q215E+H276C, T167P+Q215E+R280K, T167P+Q215E+F286C, T167P+Q215E+G366N, T167P+Q215E+D486E, T167P+H276C+R280K, T167P+H276C+F286C, T167P+H276C+G366N, T167P+H276C+D486E, T167P+R280K+F286C, T167P+R280K+G366N, T167P+R280K+D486E, T167P+F286C+G366N, T167P+F286C+D486E, T167P+G366N+D486E, Q215E+H276C+R280K, Q215E+H276C+F286C, Q215E+H276C+G366N, Q215E+H276C+D486E, Q215E+R280K+F286C, Q215E+R280K+G366N, Q215E+R280K+D486E, Q215E+F286C+G366N, Q215E+F286C+D486E, Q215E+G366N+D486E, H276C+R280K+F286C, H276C+R280K+G366N, H276C+R280K+D486E, H276C+F286C+G366N, H276C+F286C+D486E, H276C+G366N+D486E, R280K+F286C+G366N, R280K+F286C+D486E, R280K+G366N+D486E, F286C+G366N+D486E, I30L+D48P+Y155H+T167P, I30L+D48P+Y155H+Q215E, I30L+D48P+Y155H+H276C, I30L+D48P+Y155H+R280K, I30L+D48P+Y155H+F286C, I30L+D48P+Y155H+G366N, I30L+D48P+Y155H+D486E, I30L+D48P+T167P+Q215E, I30L+D48P+T167P+H276C, I30L+D48P+T167P+R280K, I30L+D48P+T167P+F286C, I30L+D48P+T167P+G366N, I30L+D48P+T167P+D486E, I30L+D48P+Q215E+H276C, I30L+D48P+Q215E+R280K, I30L+D48P+Q215E+F286C, I30L+D48P+Q215E+G366N, I30L+D48P+Q215E+D486E, I30L+D48P+H276C+R280K, I30L+D48P+H276C+F286C, I30L+D48P+H276C+G366N, I30L+D48P+H276C+D486E, I30L+D48P+R280K+F286C, I30L+D48P+R280K+G366N, I30L+D48P+R280K+D486E, I30L+D48P+F286C+G366N, I30L+D48P+F286C+D486E, I30L+D48P+G366N+D486E, I30L+Y155H+T167P+Q215E, I30L+Y155H+T167P+H276C, I30L+Y155H+T167P+R280K, I30L+Y155H+T167P+F286C, I30L+Y155H+T167P+G366N, I30L+Y155H+T167P+D486E, I30L+Y155H+Q215E+H276C, I30L+Y155H+Q215E+R280K, I30L+Y155H+Q215E+F286C, I30L+Y155H+Q215E+G366N, I30L+Y155H+Q215E+D486E, I30L+Y155H+H276C+R280K, I30L+Y155H+H276C+F286C, I30L+Y155H+H276C+G366N, I30L+Y155H+H276C+D486E, I30L+Y155H+R280K+F286C, I30L+Y155H+R280K+G366N, I30L+Y155H+R280K+D486E, I30L+Y155H+F286C+G366N, I30L+Y155H+F286C+D486E, I30L+Y155H+G366N+D486E, I30L+T167P+Q215E+H276C, I30L+T167P+Q215E+R280K, I30L+T167P+Q215E+F286C, I30L+T167P+Q215E+G366N, I30L+T167P+Q215E+D486E, I30L+T167P+H276C+R280K, I30L+T167P+H276C+F286C, I30L+T167P+H276C+G366N, I30L+T167P+H276C+D486E, I30L+T167P+R280K+F286C, I30L+T167P+R280K+G366N, I30L+T167P+R280K+D486E, I30L+T167P+F286C+G366N, I30L+T167P+F286C+D486E, I30L+T167P+G366N+D486E, I30L+Q215E+H276C+R280K, I30L+Q215E+H276C+F286C, I30L+Q215E+H276C+G366N, I30L+Q215E+H276C+D486E, I30L+Q215E+R280K+F286C, I30L+Q215E+R280K+G366N, I30L+Q215E+R280K+D486E, I30L+Q215E+F286C+G366N, I30L+Q215E+F286C+D486E, I30L+Q215E+G366N+D486E, I30L+H276C+R280K+F286C, I30L+H276C+R280K+G366N, I30L+H276C+R280K+D486E, I30L+H276C+F286C+G366N, I30L+H276C+F286C+D486E, I30L+H276C+G366N+D486E, I30L+R280K+F286C+G366N, I30L+R280K+F286C+D486E, I30L+R280K+G366N+D486E, I30L+F286C+G366N+D486E, D48P+Y155H+T167P+Q215E, D48P+Y155H+T167P+H276C, D48P+Y155H+T167P+R280K, D48P+Y155H+T167P+F286C, D48P+Y155H+T167P+G366N, D48P+Y155H+T167P+D486E, D48P+Y155H+Q215E+H276C, D48P+Y155H+Q215E+R280K, D48P+Y155H+Q215E+F286C, D48P+Y155H+Q215E+G366N, D48P+Y155H+Q215E+D486E, D48P+Y155H+H276C+R280K, D48P+Y155H+H276C+F286C, D48P+Y155H+H276C+G366N, D48P+Y155H+H276C+D486E, D48P+Y155H+R280K+F286C, D48P+Y155H+R280K+G366N, D48P+Y155H+R280K+D486E, D48P+Y155H+F286C+G366N, D48P+Y155H+F286C+D486E, D48P+Y155H+G366N+D486E, D48P+T167P+Q215E+H276C, D48P+T167P+Q215E+R280K, D48P+T167P+Q215E+F286C, D48P+T167P+Q215E+G366N, D48P+T167P+Q215E+D486E, D48P+T167P+H276C+R280K, D48P+T167P+H276C+F286C, D48P+T167P+H276C+G366N, D48P+T167P+H276C+D486E, D48P+T167P+R280K+F286C, D48P+T167P+R280K+G366N, D48P+T167P+R280K+D486E, D48P+T167P+F286C+G366N, D48P+T167P+F286C+D486E, D48P+T167P+G366N+D486E, D48P+Q215E+H276C+R280K, D48P+Q215E+H276C+F286C, D48P+Q215E+H276C+G366N, D48P+Q215E+H276C+D486E, D48P+Q215E+R280K+F286C, D48P+Q215E+R280K+G366N, D48P+Q215E+R280K+D486E, D48P+Q215E+F286C+G366N, D48P+Q215E+F286C+D486E, D48P+Q215E+G366N+D486E, D48P+H276C+R280K+F286C, D48P+H276C+R280K+G366N, D48P+H276C+R280K+D486E, D48P+H276C+F286C+G366N, D48P+H276C+F286C+D486E, D48P+H276C+G366N+D486E, D48P+R280K+F286C+G366N, D48P+R280K+F286C+D486E, D48P+R280K+G366N+D486E, D48P+F286C+G366N+D486E, Y155H+T167P+Q215E+H276C, Y155H+T167P+Q215E+R280K, Y155H+T167P+Q215E+F286C, Y155H+T167P+Q215E+G366N, Y155H+T167P+Q215E+D486E, Y155H+T167P+H276C+R280K, Y155H+T167P+H276C+F286C, Y155H+T167P+H276C+G366N, Y155H+T167P+H276C+D486E, Y155H+T167P+R280K+F286C, Y155H+T167P+R280K+G366N, Y155H+T167P+R280K+D486E, Y155H+T167P+F286C+G366N, Y155H+T167P+F286C+D486E, Y155H+T167P+G366N+D486E, Y155H+Q215E+H276C+R280K, Y155H+Q215E+H276C+F286C, Y155H+Q215E+H276C+G366N, Y155H+Q215E+H276C+D486E, Y155H+Q215E+R280K+F286C, Y155H+Q215E+R280K+G366N, Y155H+Q215E+R280K+D486E, Y155H+Q215E+F286C+G366N, Y155H+Q215E+F286C+D486E, Y155H+Q215E+G366N+D486E, Y155H+H276C+R280K+F286C, Y155H+H276C+R280K+G366N, Y155H+H276C+R280K+D486E, Y155H+H276C+F286C+G366N, Y155H+H276C+F286C+D486E, Y155H+H276C+G366N+D486E, Y155H+R280K+F286C+G366N, Y155H+R280K+F286C+D486E, Y155H+R280K+G366N+D486E, Y155H+F286C+G366N+D486E, T167P+Q215E+H276C+R280K, T167P+Q215E+H276C+F286C, T167P+Q215E+H276C+G366N, T167P+Q215E+H276C+D486E, T167P+Q215E+R280K+F286C, T167P+Q215E+R280K+G366N, T167P+Q215E+R280K+D486E, T167P+Q215E+F286C+G366N, T167P+Q215E+F286C+D486E, T167P+Q215E+G366N+D486E, T167P+H276C+R280K+F286C, T167P+H276C+R280K+G366N, T167P+H276C+R280K+D486E, T167P+H276C+F286C+G366N, T167P+H276C+F286C+D486E, T167P+H276C+G366N+D486E, T167P+R280K+F286C+G366N, T167P+R280K+F286C+D486E, T167P+R280K+G366N+D486E, T167P+F286C+G366N+D486E, Q215E+H276C+R280K+F286C, Q215E+H276C+R280K+G366N, Q215E+H276C+R280K+D486E, Q215E+H276C+F286C+G366N, Q215E+H276C+F286C+D486E, Q215E+H276C+G366N+D486E, Q215E+R280K+F286C+G366N, Q215E+R280K+F286C+D486E, Q215E+R280K+G366N+D486E, Q215E+F286C+G366N+D486E, H276C+R280K+F286C+G366N, H276C+R280K+F286C+D486E, H276C+R280K+G366N+D486E, H276C+F286C+G366N+D486E, R280K+F286C+G366N+D486E, I30L+D48P+Y155H+T167P+Q215E, I30L+D48P+Y155H+T167P+H276C, I30L+D48P+Y155H+T167P+R280K, I30L+D48P+Y155H+T167P+F286C, I30L+D48P+Y155H+T167P+G366N, I30L+D48P+Y155H+T167P+D486E, I30L+D48P+Y155H+Q215E+H276C, I30L+D48P+Y155H+Q215E+R280K, I30L+D48P+Y155H+Q215E+F286C, I30L+D48P+Y155H+Q215E+G366N, I30L+D48P+Y155H+Q215E+D486E, I30L+D48P+Y155H+H276C+R280K, I30L+D48P+Y155H+H276C+F286C, I30L+D48P+Y155H+H276C+G366N, I30L+D48P+Y155H+H276C+D486E, I30L+D48P+Y155H+R280K+F286C, I30L+D48P+Y155H+R280K+G366N, I30L+D48P+Y155H+R280K+D486E, I30L+D48P+Y155H+F286C+G366N, I30L+D48P+Y155H+F286C+D486E, I30L+D48P+Y155H+G366N+D486E, I30L+D48P+T167P+Q215E+H276C, I30L+D48P+T167P+Q215E+R280K, I30L+D48P+T167P+Q215E+F286C, I30L+D48P+T167P+Q215E+G366N, I30L+D48P+T167P+Q215E+D486E, I30L+D48P+T167P+H276C+R280K, I30L+D48P+T167P+H276C+F286C, I30L+D48P+T167P+H276C+G366N, I30L+D48P+T167P+H276C+D486E, I30L+D48P+T167P+R280K+F286C, I30L+D48P+T167P+R280K+G366N, I30L+D48P+T167P+R280K+D486E, I30L+D48P+T167P+F286C+G366N, I30L+D48P+T167P+F286C+D486E, I30L+D48P+T167P+G366N+D486E, 30L+D48P+R280K+G366N+D486E, I30L+D48P+F286C+G366N+D486E, I30L+Y155H+T167P+Q215E+H276C, I30L+Y155H+T167P+Q215E+R280K, 30L+Y155H+T167P+R280K+D486E, I30L+Y155H+T167P+F286C+G366N, I30L+Y155H+T167P+F286C+D486E, I30L+Y155H+T167P+G366N+D486E, I30L+Y155H+Q215E+H276C+R280K, I30L+Y155H+Q215E+H276C+F286C, I30L+Y155H+Q215E+H276C+G366N, I30L+Y155H+Q215E+H276C+D486E, I30L+Y155H+Q215E+R280K+F286C, I30L+Y155H+Q215E+R280K+G366N, I30L+Y155H+Q215E+R280K+D486E, I30L+Y155H+Q215E+F286C+G366N, I30L+Y155H+Q215E+F286C+D486E, I30L+Y155H+Q215E+G366N+D486E, I30L+Y155H+H276C+R280K+F286C, I30L+Y155H+H276C+R280K+G366N, I30L+Y155H+H276C+R280K+D486E, I30L+Y155H+H276C+F286C+G366N, I30L+Y155H+H276C+F286C+D486E, I30L+Y155H+H276C+G366N+D486E, I30L+Y155H+R280K+F286C+G366N, I30L+Y155H+R280K+F286C+D486E, I30L+Y155H+R280K+G366N+D486E, I30L+Y155H+F286C+G366N+D486E, I30L+T167P+Q215E+H276C+R280K, I30L+T167P+Q215E+H276C+F286C, I30L+T167P+Q215E+H276C+G366N, I30L+T167P+Q215E+H276C+D486E, I30L+T167P+Q215E+R280K+F286C, I30L+T167P+Q215E+R280K+G366N, I30L+T167P+Q215E+R280K+D486E, I30L+T167P+Q215E+F286C+G366N, I30L+T167P+Q215E+F286C+D486E, I30L+T167P+Q215E+G366N+D486E, I30L+T167P+H276C+R280K+F286C, I30L+T167P+H276C+R280K+G366N, I30L+T167P+H276C+R280K+D486E, I30L+T167P+H276C+F286C+G366N, I30L+T167P+H276C+F286C+D486E, I30L+T167P+H276C+G366N+D486E, I30L+T167P+R280K+F286C+G366N, I30L+T167P+R280K+F286C+D486E, I30L+T167P+R280K+G366N+D486E, I30L+T167P+F286C+G366N+D486E, I30L+Q215E+H276C+R280K+F286C, I30L+Q215E+H276C+R280K+G366N, I30L+Q215E+H276C+R280K+D486E, I30L+Q215E+H276C+F286C+G366N, I30L+Q215E+H276C+F286C+D486E, I30L+Q215E+H276C+G366N+D486E, I30L+Q215E+R280K+F286C+G366N, I30L+Q215E+R280K+F286C+D486E, I30L+Q215E+R280K+G366N+D486E, I30L+Q215E+F286C+G366N+D486E, I30L+H276C+R280K+F286C+G366N, I30L+H276C+R280K+F286C+D486E, I30L+H276C+R280K+G366N+D486E, I30L+H276C+F286C+G366N+D486E, I30L+R280K+F286C+G366N+D486E, D48P+Y155H+T167P+Q215E+H276C, D48P+Y155H+T167P+Q215E+R280K, D48P+Y155H+T167P+Q215E+F286C, D48P+Y155H+T167P+Q215E+G366N, D48P+Y155H+T167P+Q215E+D486E, D48P+Y155H+T167P+H276C+R280K, D48P+Y155H+T167P+H276C+F286C, D48P+Y155H+T167P+H276C+G366N, D48P+Y155H+T167P+H276C+D486E, D48P+Y155H+T167P+R280K+F286C, D48P+Y155H+T167P+R280K+G366N, D48P+Y155H+T167P+R280K+D486E, D48P+Y155H+T167P+F286C+G366N, D48P+Y155H+T167P+F286C+D486E, D48P+Y155H+T167P+G366N+D486E, D48P+Y155H+Q215E+H276C+R280K, D48P+Y155H+Q215E+H276C+F286C, D48P+Y155H+Q215E+H276C+G366N, D48P+Y155H+Q215E+H276C+D486E, D48P+Y155H+Q215E+R280K+F286C, D48P+Y155H+Q215E+R280K+G366N, D48P+Y155H+Q215E+R280K+D486E, D48P+Y155H+Q215E+F286C+G366N, D48P+Y155H+Q215E+F286C+D486E, D48P+Y155H+Q215E+G366N+D486E, D48P+Y155H+H276C+R280K+F286C, D48P+Y155H+H276C+R280K+G366N, D48P+Y155H+H276C+R280K+D486E, D48P+Y155H+H276C+F286C+G366N, D48P+Y155H+H276C+F286C+D486E, D48P+Y155H+H276C+G366N+D486E, D48P+Y155H+R280K+F286C+G366N, D48P+Y155H+R280K+F286C+D486E, D48P+Y155H+R280K+G366N+D486E, D48P+Y155H+F286C+G366N+D486E, D48P+T167P+Q215E+H276C+R280K, D48P+T167P+Q215E+H276C+F286C, D48P+T167P+Q215E+H276C+G366N, D48P+T167P+Q215E+H276C+D486E, D48P+T167P+Q215E+R280K+F286C, D48P+T167P+Q215E+R280K+G366N, D48P+T167P+Q215E+R280K+D486E, D48P+T167P+Q215E+F286C+G366N, D48P+T167P+Q215E+F286C+D486E, D48P+T167P+Q215E+G366N+D486E, D48P+T167P+H276C+R280K+F286C, D48P+T167P+H276C+R280K+G366N, D48P+T167P+H276C+R280K+D486E, D48P+T167P+H276C+F286C+G366N, D48P+T167P+H276C+F286C+D486E, D48P+T167P+H276C+G366N+D486E, D48P+T167P+R280K+F286C+G366N, D48P+T167P+R280K+F286C+D486E, D48P+T167P+R280K+G366N+D486E, D48P+T167P+F286C+G366N+D486E, D48P+Q215E+H276C+R280K+F286C, D48P+Q215E+H276C+R280K+G366N, D48P+Q215E+H276C+R280K+D486E, D48P+Q215E+H276C+F286C+G366N, D48P+Q215E+H276C+F286C+D486E, D48P+Q215E+H276C+G366N+D486E, D48P+Q215E+R280K+F286C+G366N, D48P+Q215E+R280K+F286C+D486E, D48P+Q215E+R280K+G366N+D486E, D48P+Q215E+F286C+G366N+D486E, D48P+H276C+R280K+F286C+G366N, D48P+H276C+R280K+F286C+D486E, D48P+H276C+R280K+G366N+D486E, D48P+H276C+F286C+G366N+D486E, D48P+R280K+F286C+G366N+D486E, Y155H+T167P+Q215E+H276C+R280K, Y155H+T167P+Q215E+H276C+F286C, Y155H+T167P+Q215E+H276C+G366N, Y155H+T167P+Q215E+H276C+D486E, Y155H+T167P+Q215E+R280K+F286C, Y155H+T167P+Q215E+R280K+G366N, Y155H+T167P+Q215E+R280K+D486E, Y155H+T167P+Q215E+F286C+G366N, Y155H+T167P+Q215E+F286C+D486E, Y155H+T167P+Q215E+G366N+D486E, Y155H+T167P+H276C+R280K+F286C, Y155H+T167P+H276C+R280K+G366N, Y155H+T167P+H276C+R280K+D486E, Y155H+T167P+H276C+F286C+G366N, Y155H+T167P+H276C+F286C+D486E, Y155H+T167P+H276C+G366N+D486E, Y155H+T167P+R280K+F286C+G366N, Y155H+T167P+R280K+F286C+D486E, Y155H+T167P+R280K+G366N+D486E, Y155H+T167P+F286C+G366N+D486E, Y155H+Q215E+H276C+R280K+F286C, Y155H+Q215E+H276C+R280K+G366N, Y155H+Q215E+H276C+R280K+D486E, Y155H+Q215E+H276C+F286C+G366N, Y155H+Q215E+H276C+F286C+D486E, Y155H+Q215E+H276C+G366N+D486E, Y155H+Q215E+R280K+F286C+G366N, Y155H+Q215E+R280K+F286C+D486E, Y155H+Q215E+R280K+G366N+D486E, Y155H+Q215E+F286C+G366N+D486E, Y155H+H276C+R280K+F286C+G366N, Y155H+H276C+R280K+F286C+D486E, Y155H+H276C+R280K+G366N+D486E, Y155H+H276C+F286C+G366N+D486E, Y155H+R280K+F286C+G366N+D486E, T167P+Q215E+H276C+R280K+F286C, T167P+Q215E+H276C+R280K+G366N, T167P+Q215E+H276C+R280K+D486E, T167P+Q215E+H276C+F286C+G366N, T167P+Q215E+H276C+F286C+D486E, T167P+Q215E+H276C+G366N+D486E, T167P+Q215E+R280K+F286C+G366N, T167P+Q215E+R280K+F286C+D486E, T167P+Q215E+R280K+G366N+D486E, T167P+Q215E+F286C+G366N+D486E, T167P+H276C+R280K+F286C+G366N, T167P+H276C+R280K+F286C+D486E, T167P+H276C+R280K+G366N+D486E, T167P+H276C+F286C+G366N+D486E, T167P+R280K+F286C+G366N+D486E, Q215E+H276C+R280K+F286C+G366N, Q215E+H276C+R280K+F286C+D486E, Q215E+H276C+R280K+G366N+D486E, Q215E+H276C+F286C+G366N+D486E, Q215E+R280K+F286C+G366N+D486E, H276C+R280K+F286C+G366N+D486E, I30L+D48P+Y155H+T167P+Q215E+H276C, I30L+D48P+Y155H+T167P+Q215E+R280K, I30L+D48P+Y155H+T167P+Q215E+F286C, I30L+D48P+Y155H+T167P+Q215E+G366N, I30L+D48P+Y155H+T167P+Q215E+D486E, I30L+D48P+Y155H+T167P+H276C+R280K, I30L+D48P+Y155H+T167P+H276C+F286C, I30L+D48P+Y155H+T167P+H276C+G366N, I30L+D48P+Y155H+T167P+H276C+D486E, I30L+D48P+Y155H+T167P+R280K+F286C, I30L+D48P+Y155H+T167P+R280K+G366N, I30L+D48P+Y155H+T167P+R280K+D486E, I30L+D48P+Y155H+T167P+F286C+G366N, I30L+D48P+Y155H+T167P+F286C+D486E, I30L+D48P+Y155H+T167P+G366N+D486E, I30L+D48P+Y155H+Q215E+H276C+R280K, I30L+D48P+Y155H+Q215E+H276C+F286C, I30L+D48P+Y155H+Q215E+H276C+G366N, I30L+D48P+Y155H+Q215E+H276C+D486E, I30L+D48P+Y155H+Q215E+R280K+F286C, I30L+D48P+Y155H+Q215E+R280K+G366N, I30L+D48P+Y155H+Q215E+R280K+D486E, I30L+D48P+Y155H+Q215E+F286C+G366N, I30L+D48P+Y155H+Q215E+F286C+D486E, I30L+D48P+Y155H+Q215E+G366N+D486E, I30L+D48P+Y155H+H276C+R280K+F286C, I30L+D48P+Y155H+H276C+R280K+G366N, I30L+D48P+Y155H+H276C+R280K+D486E, I30L+D48P+Y155H+H276C+F286C+G366N, I30L+D48P+Y155H+H276C+F286C+D486E, I30L+D48P+Y155H+H276C+G366N+D486E, I30L+D48P+Y155H+R280K+F286C+G366N, I30L+D48P+Y155H+R280K+F286C+D486E, I30L+D48P+Y155H+R280K+G366N+D486E, I30L+D48P+Y155H+F286C+G366N+D486E, I30L+D48P+T167P+Q215E+H276C+R280K, I30L+D48P+T167P+Q215E+H276C+F286C, I30L+D48P+T167P+Q215E+H276C+G366N, I30L+D48P+T167P+Q215E+H276C+D486E, I30L+D48P+T167P+Q215E+R280K+F286C, I30L+D48P+T167P+Q215E+R280K+G366N, I30L+D48P+T167P+Q215E+R280K+D486E, I30L+D48P+T167P+Q215E+F286C+G366N, I30L+D48P+T167P+Q215E+F286C+D486E, I30L+D48P+T167P+Q215E+G366N+D486E, I30L+D48P+T167P+H276C+R280K+F286C, I30L+D48P+T167P+H276C+R280K+G366N, I30L+D48P+T167P+H276C+R280K+D486E, I30L+D48P+T167P+H276C+F286C+G366N, I30L+D48P+T167P+H276C+F286C+D486E, I30L+D48P+T167P+H276C+G366N+D486E, I30L+D48P+T167P+R280K+F286C+G366N, I30L+D48P+T167P+R280K+F286C+D486E, I30L+D48P+T167P+R280K+G366N+D486E, I30L+D48P+T167P+F286C+G366N+D486E, I30L+D48P+Q215E+H276C+R280K+F286C, I30L+D48P+Q215E+H276C+R280K+G366N, I30L+D48P+Q215E+H276C+R280K+D486E, I30L+D48P+Q215E+H276C+F286C+G366N, I30L+D48P+Q215E+H276C+F286C+D486E, I30L+D48P+Q215E+H276C+G366N+D486E, I30L+D48P+Q215E+R280K+F286C+G366N, I30L+D48P+Q215E+R280K+F286C+D486E, I30L+D48P+Q215E+R280K+G366N+D486E, I30L+D48P+Q215E+F286C+G366N+D486E, I30L+D48P+H276C+R280K+F286C+G366N, I30L+D48P+H276C+R280K+F286C+D486E, I30L+D48P+H276C+R280K+G366N+D486E, I30L+D48P+H276C+F286C+G366N+D486E, I30L+D48P+R280K+F286C+G366N+D486E, I30L+Y155H+T167P+Q215E+H276C+R280K, I30L+Y155H+T167P+Q215E+H276C+F286C, I30L+Y155H+T167P+Q215E+H276C+G366N, I30L+Y155H+T167P+Q215E+H276C+D486E, I30L+Y155H+T167P+Q215E+R280K+F286C, I30L+Y155H+T167P+Q215E+R280K+G366N, I30L+Y155H+T167P+Q215E+R280K+D486E, I30L+Y155H+T167P+Q215E+F286C+G366N, I30L+Y155H+T167P+Q215E+F286C+D486E, I30L+Y155H+T167P+Q215E+G366N+D486E, I30L+Y155H+T167P+H276C+R280K+F286C, I30L+Y155H+T167P+H276C+R280K+G366N, I30L+Y155H+T167P+H276C+R280K+D486E, I30L+Y155H+T167P+H276C+F286C+G366N, I30L+Y155H+T167P+H276C+F286C+D486E, I30L+Y155H+T167P+H276C+G366N+D486E, I30L+Y155H+T167P+R280K+F286C+G366N, I30L+Y155H+T167P+R280K+F286C+D486E, I30L+Y155H+T167P+R280K+G366N+D486E, I30L+Y155H+T167P+F286C+G366N+D486E, I30L+Y155H+Q215E+H276C+R280K+F286C, I30L+Y155H+Q215E+H276C+R280K+G366N, I30L+Y155H+Q215E+H276C+R280K+D486E, I30L+Y155H+Q215E+H276C+F286C+G366N, I30L+Y155H+Q215E+H276C+F286C+D486E, I30L+Y155H+Q215E+H276C+G366N+D486E, I30L+Y155H+Q215E+R280K+F286C+G366N, I30L+Y155H+Q215E+R280K+F286C+D486E, I30L+Y155H+Q215E+R280K+G366N+D486E, I30L+Y155H+Q215E+F286C+G366N+D486E, I30L+Y155H+H276C+R280K+F286C+G366N, I30L+Y155H+H276C+R280K+F286C+D486E, I30L+Y155H+H276C+R280K+G366N+D486E, I30L+Y155H+H276C+F286C+G366N+D486E, I30L+Y155H+R280K+F286C+G366N+D486E, I30L+T167P+Q215E+H276C+R280K+F286C, I30L+T167P+Q215E+H276C+R280K+G366N, I30L+T167P+Q215E+H276C+R280K+D486E, I30L+T167P+Q215E+H276C+F286C+G366N, I30L+T167P+Q215E+H276C+F286C+D486E, I30L+T167P+Q215E+H276C+G366N+D486E, I30L+T167P+Q215E+R280K+F286C+G366N, I30L+T167P+Q215E+R280K+F286C+D486E, I30L+T167P+Q215E+R280K+G366N+D486E, I30L+T167P+Q215E+F286C+G366N+D486E, I30L+T167P+H276C+R280K+F286C+G366N, I30L+T167P+H276C+R280K+F286C+D486E, I30L+T167P+H276C+R280K+G366N+D486E, I30L+T167P+H276C+F286C+G366N+D486E, I30L+T167P+R280K+F286C+G366N+D486E, I30L+Q215E+H276C+R280K+F286C+G366N, I30L+Q215E+H276C+R280K+F286C+D486E, I30L+Q215E+H276C+R280K+G366N+D486E, I30L+Q215E+H276C+F286C+G366N+D486E, I30L+Q215E+R280K+F286C+G366N+D486E, I30L+H276C+R280K+F286C+G366N+D486E, D48P+Y155H+T167P+Q215E+H276C+R280K, D48P+Y155H+T167P+Q215E+H276C+F286C, D48P+Y155H+T167P+Q215E+H276C+G366N, D48P+Y155H+T167P+Q215E+H276C+D486E, D48P+Y155H+T167P+Q215E+R280K+F286C, D48P+Y155H+T167P+Q215E+R280K+G366N, D48P+Y155H+T167P+Q215E+R280K+D486E, D48P+Y155H+T167P+Q215E+F286C+G366N, D48P+Y155H+T167P+Q215E+F286C+D486E, D48P+Y155H+T167P+Q215E+G366N+D486E, D48P+Y155H+T167P+H276C+R280K+F286C, D48P+Y155H+T167P+H276C+R280K+G366N, D48P+Y155H+T167P+H276C+R280K+D486E, D48P+Y155H+T167P+H276C+F286C+G366N, D48P+Y155H+T167P+H276C+F286C+D486E, D48P+Y155H+T167P+H276C+G366N+D486E, D48P+Y155H+T167P+R280K+F286C+G366N, D48P+Y155H+T167P+R280K+F286C+D486E, D48P+Y155H+T167P+R280K+G366N+D486E, D48P+Y155H+T167P+F286C+G366N+D486E, D48P+Y155H+Q215E+H276C+R280K+F286C, D48P+Y155H+Q215E+H276C+R280K+G366N, D48P+Y155H+Q215E+H276C+R280K+D486E, D48P+Y155H+Q215E+H276C+F286C+G366N, D48P+Y155H+Q215E+H276C+F286C+D486E, D48P+Y155H+Q215E+H276C+G366N+D486E, D48P+Y155H+Q215E+R280K+F286C+G366N, D48P+Y155H+Q215E+R280K+F286C+D486E, D48P+Y155H+Q215E+R280K+G366N+D486E, D48P+Y155H+Q215E+F286C+G366N+D486E, D48P+Y155H+H276C+R280K+F286C+G366N, D48P+Y155H+H276C+R280K+F286C+D486E, D48P+Y155H+H276C+R280K+G366N+D486E, D48P+Y155H+H276C+F286C+G366N+D486E, D48P+Y155H+R280K+F286C+G366N+D486E, D48P+T167P+Q215E+H276C+R280K+F286C, D48P+T167P+Q215E+H276C+R280K+G366N, D48P+T167P+Q215E+H276C+R280K+D486E, D48P+T167P+Q215E+H276C+F286C+G366N, D48P+T167P+Q215E+H276C+F286C+D486E, D48P+T167P+Q215E+H276C+G366N+D486E, D48P+T167P+Q215E+R280K+F286C+G366N, D48P+T167P+Q215E+R280K+F286C+D486E, D48P+T167P+Q215E+R280K+G366N+D486E, D48P+T167P+Q215E+F286C+G366N+D486E, D48P+T167P+H276C+R280K+F286C+G366N, D48P+T167P+H276C+R280K+F286C+D486E, D48P+T167P+H276C+R280K+G366N+D486E, D48P+T167P+H276C+F286C+G366N+D486E, D48P+T167P+R280K+F286C+G366N+D486E, D48P+Q215E+H276C+R280K+F286C+G366N, D48P+Q215E+H276C+R280K+F286C+D486E, D48P+Q215E+H276C+R280K+G366N+D486E, D48P+Q215E+H276C+F286C+G366N+D486E, D48P+Q215E+R280K+F286C+G366N+D486E, D48P+H276C+R280K+F286C+G366N+D486E, Y155H+T167P+Q215E+H276C+R280K+F286C, Y155H+T167P+Q215E+H276C+R280K+G366N, Y155H+T167P+Q215E+H276C+R280K+D486E, Y155H+T167P+Q215E+H276C+F286C+G366N, Y155H+T167P+Q215E+H276C+F286C+D486E, Y155H+T167P+Q215E+H276C+G366N+D486E, Y155H+T167P+Q215E+R280K+F286C+G366N, Y155H+T167P+Q215E+R280K+F286C+D486E, Y155H+T167P+Q215E+R280K+G366N+D486E, Y155H+T167P+Q215E+F286C+G366N+D486E, Y155H+T167P+H276C+R280K+F286C+G366N, Y155H+T167P+H276C+R280K+F286C+D486E, Y155H+T167P+H276C+R280K+G366N+D486E, Y155H+T167P+H276C+F286C+G366N+D486E, Y155H+T167P+R280K+F286C+G366N+D486E, Y155H+Q215E+H276C+R280K+F286C+G366N, Y155H+Q215E+H276C+R280K+F286C+D486E, Y155H+Q215E+H276C+R280K+G366N+D486E, Y155H+Q215E+H276C+F286C+G366N+D486E, Y155H+Q215E+R280K+F286C+G366N+D486E, Y155H+H276C+R280K+F286C+G366N+D486E, T167P+Q215E+H276C+R280K+F286C+G366N, T167P+Q215E+H276C+R280K+F286C+D486E, T167P+Q215E+H276C+R280K+G366N+D486E, T167P+Q215E+H276C+F286C+G366N+D486E, T167P+Q215E+R280K+F286C+G366N+D486E, T167P+H276C+R280K+F286C+G366N+D486E, Q215E+H276C+R280K+F286C+G366N+D486E, I30L+D48P+Y155H+T167P+Q215E+H276C+R280K, I30L+D48P+Y155H+T167P+Q215E+H276C+F286C, I30L+D48P+Y155H+T167P+Q215E+H276C+G366N, I30L+D48P+Y155H+T167P+Q215E+H276C+D486E, I30L+D48P+Y155H+T167P+Q215E+R280K+F286C, I30L+D48P+Y155H+T167P+Q215E+R280K+G366N, I30L+D48P+Y155H+T167P+Q215E+R280K+D486E, I30L+D48P+Y155H+T167P+Q215E+F286C+G366N, I30L+D48P+Y155H+T167P+Q215E+F286C+D486E, I30L+D48P+Y155H+T167P+Q215E+G366N+D486E, I30L+D48P+Y155H+T167P+H276C+R280K+F286C, I30L+D48P+Y155H+T167P+H276C+R280K+G366N, I30L+D48P+Y155H+T167P+H276C+R280K+D486E, I30L+D48P+Y155H+T167P+H276C+F286C+G366N, I30L+D48P+Y155H+T167P+H276C+F286C+D486E, I30L+D48P+Y155H+T167P+H276C+G366N+D486E, I30L+D48P+Y155H+T167P+R280K+F286C+G366N, I30L+D48P+Y155H+T167P+R280K+F286C+D486E, I30L+D48P+Y155H+T167P+R280K+G366N+D486E, I30L+D48P+Y155H+T167P+F286C+G366N+D486E, I30L+D48P+Y155H+Q215E+H276C+R280K+F286C, I30L+D48P+Y155H+Q215E+H276C+R280K+G366N, I30L+D48P+Y155H+Q215E+H276C+R280K+D486E, I30L+D48P+Y155H+Q215E+H276C+F286C+G366N, I30L+D48P+Y155H+Q215E+H276C+F286C+D486E, I30L+D48P+Y155H+Q215E+H276C+G366N+D486E, I30L+D48P+Y155H+Q215E+R280K+F286C+G366N, I30L+D48P+Y155H+Q215E+R280K+F286C+D486E, I30L+D48P+Y155H+Q215E+R280K+G366N+D486E, I30L+D48P+Y155H+Q215E+F286C+G366N+D486E, I30L+D48P+Y155H+H276C+R280K+F286C+G366N, I30L+D48P+Y155H+H276C+R280K+F286C+D486E, I30L+D48P+Y155H+H276C+R280K+G366N+D486E, I30L+D48P+Y155H+H276C+F286C+G366N+D486E, I30L+D48P+Y155H+R280K+F286C+G366N+D486E, I30L+D48P+T167P+Q215E+H276C+R280K+F286C, I30L+D48P+T167P+Q215E+H276C+R280K+G366N, I30L+D48P+T167P+Q215E+H276C+R280K+D486E, I30L+D48P+T167P+Q215E+H276C+F286C+G366N, I30L+D48P+T167P+Q215E+H276C+F286C+D486E, I30L+D48P+T167P+Q215E+H276C+G366N+D486E, I30L+D48P+T167P+Q215E+R280K+F286C+G366N, I30L+D48P+T167P+Q215E+R280K+F286C+D486E, I30L+D48P+T167P+Q215E+R280K+G366N+D486E, I30L+D48P+T167P+Q215E+F286C+G366N+D486E, I30L+D48P+T167P+H276C+R280K+F286C+G366N, I30L+D48P+T167P+H276C+R280K+F286C+D486E, I30L+D48P+T167P+H276C+R280K+G366N+D486E, I30L+D48P+T167P+H276C+F286C+G366N+D486E, I30L+D48P+T167P+R280K+F286C+G366N+D486E, I30L+D48P+Q215E+H276C+R280K+F286C+G366N, I30L+D48P+Q215E+H276C+R280K+F286C+D486E, I30L+D48P+Q215E+H276C+R280K+G366N+D486E, I30L+D48P+Q215E+H276C+F286C+G366N+D486E, I30L+D48P+Q215E+R280K+F286C+G366N+D486E, I30L+D48P+H276C+R280K+F286C+G366N+D486E, I30L+Y155H+T167P+Q215E+H276C+R280K+F286C, I30L+Y155H+T167P+Q215E+H276C+R280K+G366N, I30L+Y155H+T167P+Q215E+H276C+R280K+D486E, I30L+Y155H+T167P+Q215E+H276C+F286C+G366N, I30L+Y155H+T167P+Q215E+H276C+F286C+D486E, I30L+Y155H+T167P+Q215E+H276C+G366N+D486E, I30L+Y155H+T167P+Q215E+R280K+F286C+G366N, I30L+Y155H+T167P+Q215E+R280K+F286C+D486E, I30L+Y155H+T167P+Q215E+R280K+G366N+D486E, I30L+Y155H+T167P+Q215E+F286C+G366N+D486E, I30L+Y155H+T167P+H276C+R280K+F286C+G366N, I30L+Y155H+T167P+H276C+R280K+F286C+D486E, I30L+Y155H+T167P+H276C+R280K+G366N+D486E, I30L+Y155H+T167P+H276C+F286C+G366N+D486E, I30L+Y155H+T167P+R280K+F286C+G366N+D486E, I30L+Y155H+Q215E+H276C+R280K+F286C+G366N, I30L+Y155H+Q215E+H276C+R280K+F286C+D486E, I30L+Y155H+Q215E+H276C+R280K+G366N+D486E, I30L+Y155H+Q215E+H276C+F286C+G366N+D486E, I30L+Y155H+Q215E+R280K+F286C+G366N+D486E, I30L+Y155H+H276C+R280K+F286C+G366N+D486E, I30L+T167P+Q215E+H276C+R280K+F286C+G366N, I30L+T167P+Q215E+H276C+R280K+F286C+D486E, I30L+T167P+Q215E+H276C+R280K+G366N+D486E, I30L+T167P+Q215E+H276C+F286C+G366N+D486E, I30L+T167P+Q215E+R280K+F286C+G366N+D486E, I30L+T167P+H276C+R280K+F286C+G366N+D486E, I30L+Q215E+H276C+R280K+F286C+G366N+D486E, D48P+Y155H+T167P+Q215E+H276C+R280K+F286C, D48P+Y155H+T167P+Q215E+H276C+R280K+G366N, D48P+Y155H+T167P+Q215E+H276C+R280K+D486E, D48P+Y155H+T167P+Q215E+H276C+F286C+G366N, D48P+Y155H+T167P+Q215E+H276C+F286C+D486E, D48P+Y155H+T167P+Q215E+H276C+G366N+D486E, D48P+Y155H+T167P+Q215E+R280K+F286C+G366N, D48P+Y155H+T167P+Q215E+R280K+F286C+D486E, D48P+Y155H+T167P+Q215E+R280K+G366N+D486E, D48P+Y155H+T167P+Q215E+F286C+G366N+D486E, D48P+Y155H+T167P+H276C+R280K+F286C+G366N, D48P+Y155H+T167P+H276C+R280K+F286C+D486E, D48P+Y155H+T167P+H276C+R280K+G366N+D486E, D48P+Y155H+T167P+H276C+F286C+G366N+D486E, D48P+Y155H+T167P+R280K+F286C+G366N+D486E, D48P+Y155H+Q215E+H276C+R280K+F286C+G366N, D48P+Y155H+Q215E+H276C+R280K+F286C+D486E, D48P+Y155H+Q215E+H276C+R280K+G366N+D486E, D48P+Y155H+Q215E+H276C+F286C+G366N+D486E, D48P+Y155H+Q215E+R280K+F286C+G366N+D486E, D48P+Y155H+H276C+R280K+F286C+G366N+D486E, D48P+T167P+Q215E+H276C+R280K+F286C+G366N, D48P+T167P+Q215E+H276C+R280K+F286C+D486E, D48P+T167P+Q215E+H276C+R280K+G366N+D486E, D48P+T167P+Q215E+H276C+F286C+G366N+D486E, D48P+T167P+Q215E+R280K+F286C+G366N+D486E, D48P+T167P+H276C+R280K+F286C+G366N+D486E, D48P+Q215E+H276C+R280K+F286C+G366N+D486E, Y155H+T167P+Q215E+H276C+R280K+F286C+G366N, Y155H+T167P+Q215E+H276C+R280K+F286C+D486E, Y155H+T167P+Q215E+H276C+R280K+G366N+D486E, Y155H+T167P+Q215E+H276C+F286C+G366N+D486E, Y155H+T167P+Q215E+R280K+F286C+G366N+D486E, Y155H+T167P+H276C+R280K+F286C+G366N+D486E, Y155H+Q215E+H276C+R280K+F286C+G366N+D486E, T167P+Q215E+H276C+R280K+F286C+G366N+D486E, I30L+D48P+Y155H+T167P+Q215E+H276C+R280K+F286C, I30L+D48P+Y155H+T167P+Q215E+H276C+R280K+G366N, I30L+D48P+Y155H+T167P+Q215E+H276C+R280K+D486E, I30L+D48P+Y155H+T167P+Q215E+H276C+F286C+G366N, I30L+D48P+Y155H+T167P+Q215E+H276C+F286C+D486E, I30L+D48P+Y155H+T167P+Q215E+H276C+G366N+D486E, I30L+D48P+Y155H+T167P+Q215E+R280K+F286C+G366N, I30L+D48P+Y155H+T167P+Q215E+R280K+F286C+D486E, I30L+D48P+Y155H+T167P+Q215E+R280K+G366N+D486E, I30L+D48P+Y155H+T167P+Q215E+F286C+G366N+D486E, I30L+D48P+Y155H+T167P+H276C+R280K+F286C+G366N, I30L+D48P+Y155H+T167P+H276C+R280K+F286C+D486E, I30L+D48P+Y155H+T167P+H276C+R280K+G366N+D486E, I30L+D48P+Y155H+T167P+H276C+F286C+G366N+D486E, I30L+D48P+Y155H+T167P+R280K+F286C+G366N+D486E, I30L+D48P+Y155H+Q215E+H276C+R280K+F286C+G366N, I30L+D48P+Y155H+Q215E+H276C+R280K+F286C+D486E, I30L+D48P+Y155H+Q215E+H276C+R280K+G366N+D486E, I30L+D48P+Y155H+Q215E+H276C+F286C+G366N+D486E, I30L+D48P+Y155H+Q215E+R280K+F286C+G366N+D486E, I30L+D48P+Y155H+H276C+R280K+F286C+G366N+D486E, I30L+D48P+T167P+Q215E+H276C+R280K+F286C+G366N, I30L+D48P+T167P+Q215E+H276C+R280K+F286C+D486E, I30L+D48P+T167P+Q215E+H276C+R280K+G366N+D486E, I30L+D48P+T167P+Q215E+H276C+F286C+G366N+D486E, I30L+D48P+T167P+Q215E+R280K+F286C+G366N+D486E, I30L+D48P+T167P+H276C+R280K+F286C+G366N+D486E, I30L+D48P+Q215E+H276C+R280K+F286C+G366N+D486E, I30L+Y155H+T167P+Q215E+H276C+R280K+F286C+G366N, I30L+Y155H+T167P+Q215E+H276C+R280K+F286C+D486E, I30L+Y155H+T167P+Q215E+H276C+R280K+G366N+D486E, I30L+Y155H+T167P+Q215E+H276C+F286C+G366N+D486E, I30L+Y155H+T167P+Q215E+R280K+F286C+G366N+D486E, I30L+Y155H+T167P+H276C+R280K+F286C+G366N+D486E, I30L+Y155H+Q215E+H276C+R280K+F286C+G366N+D486E, I30L+T167P+Q215E+H276C+R280K+F286C+G366N+D486E, D48P+Y155H+T167P+Q215E+H276C+R280K+F286C+G366N, D48P+Y155H+T167P+Q215E+H276C+R280K+F286C+D486E, D48P+Y155H+T167P+Q215E+H276C+R280K+G366N+D486E, D48P+Y155H+T167P+Q215E+H276C+F286C+G366N+D486E, D48P+Y155H+T167P+Q215E+R280K+F286C+G366N+D486E, D48P+Y155H+T167P+H276C+R280K+F286C+G366N+D486E, D48P+Y155H+Q215E+H276C+R280K+F286C+G366N+D486E, D48P+T167P+Q215E+H276C+R280K+F286C+G366N+D486E, Y155H+T167P+Q215E+H276C+R280K+F286C+G366N+D486E, I30L+D48P+Y155H+T167P+Q215E+H276C+R280K+F286C+G366N, I30L+D48P+Y155H+T167P+Q215E+H276C+R280K+F286C+D486E, I30L+D48P+Y155H+T167P+Q215E+H276C+R280K+G366N+D486E, I30L+D48P+Y155H+T167P+Q215E+H276C+F286C+G366N+D486E, I30L+D48P+Y155H+T167P+Q215E+R280K+F286C+G366N+D486E, I30L+D48P+Y155H+T167P+H276C+R280K+F286C+G366N+D486E, I30L+D48P+Y155H+Q215E+H276C+R280K+F286C+G366N+D486E, I30L+D48P+T167P+Q215E+H276C+R280K+F286C+G366N+D486E, I30L+Y155H+T167P+Q215E+H276C+R280K+F286C+G366N+D486E, D48P+Y155H+T167P+Q215E+H276C+R280K+F286C+G366N+D486E, I30L+D48P+Y155H+T167P+Q215E+H276C+R280K+F286C+G366N+D486E.

In an embodiment the variant comprises the afore mentioned deletions of the amino acids corresponding to the amino acids at position 490 and 491 of SEQ ID NO:2, optionally the substitution D16A and one or more substitutions, such as 2, 3, 4, 5, 6, 7, 8, 9, 10, 11, 12, 13, 14, 15, 16, 17, 18, 19, 20, 21, 22, 22, 24 or 25 substitutions, selected from the group consisting of G20P, I30L, D48P, A101L, A111P, A118E, S137A, Q143R, Y155H, R160L, E162D, T167P, M171I, N176S, P182R, Q183E, Q215E, V244A, H276C, R280K, F286C, H324K, G366N, D385H and D486E.

In an embodiment the variant of SEQ ID NO:2 comprises the substitutions I30L, D48P, Y155H, T167P, Q215E, H276C, R280K, F286C, G366N, and D486E, and the deletions W490* and R491*.

In an embodiment the variant of SEQ ID NO:2 comprises the substitutions G20P, I30L, D48P, A101L, A111P, A118E, S137A, Q143R, Y155H, R160L, E162D, T167P, M171I, N176S, P182R, Q183E, Q215E, V244A, H276C, R280K, F286C, H324K, G366N, D385H and D486E, and the deletions W490* and R491*.

In an embodiment the variant of SEQ ID NO:2 comprises the substitutions G20P, I30L, D48P, A101L, A111P, A118E, S137A, Q143R, Y155H, R160L, E162D, T167P, M171I, N176S, P182R, Q183E, Q215E, V244A, H276C, R280K, F286C, H324K, G366N, D385H and D486E, and the deletions W490* and R491* together with at least one substitution selected from the group consisting of V161A, R164S, R164P, R164A, R164K, I165G, I165A, I165P, I165T, and I165E.

In an embodiment, the variant has a sequence identity of at least at least 90%, at least 90.1%, at least 90.2%, at least 90.3%, at least 90.4%, at least 90.5%, at least 90.6%, at least 90.7%, at least 90.8%, at least 90.9%, such as at least 91%, at least 91.1%, at least 91.2%, at least 91.3%, at least 91.4%, at least 91.5%, at least 91.6%, at least 91.7%, at least 91.8%, at least 91.9%, such as at least 92%, at least 92.1%, at least 92.2%, at least 92.3%, at least 92.4%, at least 92.5%, at least 92.6%, at least 92.7%, at least 92.8%, at least 92.9%, such as at least 93%, at least 93.1%, at least 93.2%, at least 93.3%, at least 93.4%, at least 93.5%, at least 93.6%, at least 93.7%, at least 93.8%, at least 93.9%, such as at least 94%, at least 94.1%, at least 94.2%, at least 94.3%, at least 94.4%, at least 94.5%, at least 94.6%, at least 94.7%, at least 94.8%, at least 94.9%, such as at least 95%, at least 95.1%, at least 95.2%, at least 95.3%, at least 95.4%, at least 95.5%, at least 95.6%, at least 95.7%, at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, such as at least 97%, at least 97.1%, at least 97.2%, at least 97.3%, at least 97.4%, at least 97.5%, at least 97.6%, at least 97.7%, at least 97.8%, at least 97.9%, such as at least 98%, at least 98.1%, at least 98.2%, at least 98.3%, at least 98.4%, at least 98.5%, at least 98.6%, at least 98.7%, at least 98.8%, at least 98.9%, such as at least 99%, at least 99.1%, at least 99.2%, at least 99.3%, at least 99.4%, at least 99.5%, at least 99.6%, at least 99.7%, at least 99.8%, or 99.9% sequence identity or even 100% sequence identity to the polypeptide of SEQ ID NO: 3, the polypeptide of SEQ ID NO: 4, the polypeptide of SEQ ID NO: 5, the polypeptide of SEQ ID NO: 6, the polypeptide of SEQ ID NO: 7, the polypeptide of SEQ ID NO: 22, the polypeptide of SEQ ID NO: 23, the polypeptide of SEQ ID NO: 24, the polypeptide of SEQ ID NO: 25, the polypeptide of SEQ ID NO: 26, the polypeptide of SEQ ID NO: 27, the polypeptide of SEQ ID NO: 28, or the polypeptide of SEQ ID NO: 29, wherein the variant has mannanase activity.

In an additional embodiment the amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, LeuNal, Ala/Glu, and Asp/Gly.

The variants may consist of 474 to 489 amino acids, such as 474 to 485 amino acids, such as 474, 475, 476, 477, 478, 479, 480, 481, 482, 483, 484, 485, 486, 487, 488 or 489 amino acids.

In an embodiment, the variant has improved stability in detergent under storage conditions compared to the parent enzyme as described in Example 3.

The mannanase variants of the invention are preferably isolated, more preferably the variants are purified, using standard protein purification methods known in the art.

### Preparation of Variants

The present invention also relates to methods for obtaining a variant having mannase activity with the substitutions and deletions disclosed herein. In one embodiment the method comprises: (a) introducing into a parent mannanase a deletion at position 490 and 491, and a deletion or substitution at one or more positions corresponding to positions selected from the group consisting of 1, 2, 3, 4, 5, 6, 7, 8, 9, 10, 11, 12, 13, 14, 15, 16, 20, 26, 30, 36, 46, 48, 53, 61, 64, 65, 69, 70, 74, 76, 78, 82, 101, 103, 109, 111, 112, 118, 120, 126, 137, 139, 141, 143, 155, 160, 161, 162, 163, 164, 165, 166, 167, 168, 171, 172, 176, 178, 181, 182, 183, 190, 197, 214, 215, 219, 239, 244, 248, 253, 258, 271, 276, 280, 283, 286, 299, 315, 324, 366, 378, 385, 408, 410, 413, 473, 485, and 486 of the polypeptide of SEQ ID NO: 2, wherein the variant has mannanase activity; and (b) recovering the variant.

In particular the invention relates to methods for obtaining a variant having mannase activity disclosed in the paragraph Variants above.

In another particular embodiment the invention relates to methods for obtaining a variant having mannanase activity, wherein the variant has at least 60%, e.g., at 65%, 70%, 75%, 80%, 85%, 85.5%, 86%, 86.5%, 87%, 87.5%, 88%, 88.5%, 89%, 89.5%, 90%, 90.5%, at least 90.6%, at least 90.7%, at least 90.8%, at least 90.9%, such as at least 91%, at least 91.1%, at least 91.2%, at least 91.3%, at least 91.4%, at least 91.5%, at least 91.6%, at least 91.7%, at least 91.8%, at least 91.9%, such as at least 92%, at least 92.1%, at least 92.2%, at least 92.3%, at least 92.4%, at least 92.5%, at least 92.6%, at least 92.7%, at least 92.8%, at least 92.9%, such as at least 93%, at least 93.1%, at least 93.2%, at least 93.3%, at least 93.4%, at least 93.5%, at least 93.6%, at least 93.7%, at least 93.8%, at least 93.9%, such as at least 94%, at least 94.1%, at least 94.2%, at least 94.3%, at least 94.4%, at least 94.5%, at least 94.6%, at least 94.7%, at least 94.8%, at least 94.9%, such as at least 95%, at least 95.1%, at least 95.2%, at least 95.3%, at least 95.4%, at least 95.5%, at least 95.6%, at least 95.7%, at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, such as at least 97%, at least 97.1%, at least 97.2%, at least 97.3%, at least 97.4%, at least 97.5%, at least 97.6%, at least 97.7%, at least 97.8%, at least 97.9%, such as at least 98%, at least 98.1%, at least 98.2%, at least 98.3%, at least 98.4%, at least 98.5%, at least 98.6%, at least 98.7%, at least 98.8%, at least 98.9%, such as at least 99%, at least 99.1%, at least 99.2%, at least 99.3%, at least 99.4%, at least 99.5%, at least 99.6%, at least 99.7%, at least 99.8%, at least 99.9% sequence identity or even 100% sequence identity to the polypeptide of SEQ ID NO: 3, the polypeptide of SEQ ID NO: 4, the polypeptide of SEQ ID NO: 5, the polypeptide of SEQ ID NO: 6, the polypeptide of SEQ ID NO: 7, the polypeptide of SEQ ID NO: 22, the polypeptide of SEQ ID NO: 23, the polypeptide of SEQ ID NO: 24, the polypeptide of SEQ ID NO: 25, the polypeptide of SEQ ID NO: 26, the polypeptide of SEQ ID NO: 27, the polypeptide of SEQ ID NO: 28, or the polypeptide of SEQ ID NO: 29.

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., US 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al., 2004, Nature 432: 1050-1054, and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Polynucleotides

The present invention also relates to polynucleotides encoding a variant of the present invention.

The polynucleotide may be a genomic DNA, a cDNA, a synthetic DNA, a synthetic RNA, a mRNA, or a combination thereof.

In an aspect, the polynucleotide is isolated.

In another aspect, the polynucleotide is purified.

### Nucleic Acid Constructs

The present invention also relates to nucleic acid constructs comprising a polynucleotide encoding a variant of the present invention operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

### Promoters

The control sequence may be a promoter, a polynucleotide recognized by a host cell for expression of a polynucleotide encoding a variant of the present invention. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are described in Sambrook et al., 1989, Molecular Cloning: A Laboratory Manual, Cold Spring Harbor Lab., NY, Davis et al., 2012, Basic Methods in Molecular Biology, Elsevier, and Song et al., 2016, PLOS One 11(7): e0158447.

### Terminators

The control sequence may also be a transcription terminator, which is recognized by a host cell to terminate transcription. The terminator is operably linked to the 3′-terminus of the polynucleotide encoding the variant. Any terminator that is functional in the host cell may be used in the present invention.

Preferred terminators for bacterial host cells may be obtained from the genes for Bacillus clausii alkaline protease (aprH), Bacillus licheniformis alpha-amylase (amyL), and Escherichia coli ribosomal RNA (rrnB).

mRNA Stabilizers

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177: 3465-3471).

Examples of mRNA stabilizer regions for fungal cells are described in Geisberg et al., 2014, Cell 156(4): 812-824, and in Morozov et al., 2006, Eukaryotic Cell 5(11): 1838-1846.

### Leader Sequences

The control sequence may also be a leader, a nontranslated region of an mRNA that is important for translation by the host cell. The leader is operably linked to the 5′-terminus of the polynucleotide encoding the variant. Any leader that is functional in the host cell may be used.

Suitable leaders for bacterial host cells are described by Hambraeus et al., 2000, Microbiology 146(12): 3051-3059, and by Kaberdin and Blssi, 2006, FEMS Microbiol. Rev. 30(6): 967-979.

### Polyadenylation Sequences

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

### Signal Peptides

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

### Propeptides

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active variant by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a variant and the signal peptide sequence is positioned next to the N-terminus of the propeptide sequence.

### Regulatory Sequences

It may also be desirable to add regulatory sequences that regulate expression of the variant relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems.

### Transcription Factors

The control sequence may also be a transcription factor, a polynucleotide encoding a polynucleotide-specific DNA-binding polypeptide that controls the rate of the transcription of genetic information from DNA to mRNA by binding to a specific polynucleotide sequence. The transcription factor may function alone and/or together with one or more other polypeptides or transcription factors in a complex by promoting or blocking the recruitment of RNA polymerase. Transcription factors are characterized by comprising at least one DNA-binding domain which often attaches to a specific DNA sequence adjacent to the genetic elements which are regulated by the transcription factor. The transcription factor may regulate the expression of a protein of interest either directly, i.e., by activating the transcription of the gene encoding the protein of interest by binding to its promoter, or indirectly, i.e., by activating the transcription of a further transcription factor which regulates the transcription of the gene encoding the protein of interest, such as by binding to the promoter of the further transcription factor. Suitable transcription factors for fungal host cells are described in WO 2017/144177. Suitable transcription factors for prokaryotic host cells are described in Seshasayee et al., 2011, Subcellular Biochemistry 52: 7-23, as well in Balleza et al., 2009, FEMS Microbiol. Rev. 33(1): 133-151.

### Expression Vectors

The present invention also relates to recombinant expression vectors comprising a polynucleotide encoding a variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

The vector preferably contains at least one element that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous recombination, such as homology-directed repair (HDR), or non-homologous recombination, such as non-homologous end-joining (NHEJ).

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

More than one copy of a polynucleotide of the present invention may be inserted into a host cell to increase production of a polypeptide. For example, 2 or 3 or 4 or 5 or more copies are inserted into a host cell. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

### Host Cells

The present invention also relates to recombinant host cells, comprising a polynucleotide of the present invention operably linked to one or more control sequences that direct the production of a variant of the present invention.

A construct or vector comprising a polynucleotide is introduced into a host cell so that the construct or vector is maintained as a chromosomal integrant or as a self-replicating extra-chromosomal vector as described earlier. The choice of a host cell will to a large extent depend upon the gene encoding the variant and its source. The recombinant host cell may comprise a single copy, or at least two copies, e.g., three, four, five, or more copies of the polynucleotide of the present invention.

The host cell may be any cell useful in the recombinant production of a variant of the invention, e.g., a prokaryotic cell or a fungal cell.

The host cell may be any microbial cell useful in the recombinant production of a polypeptide of the present invention, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. In an embodiment, the Bacillus cell is a Bacillus amyloliquefaciens, Bacillus licheniformis and Bacillus subtilis cell.

For purposes of this invention, Bacillus classes/genera/species shall be defined as described in Patel and Gupta, 2020, Int. J. Syst. Evol. Microbiol. 70: 406-438.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

Methods for introducing DNA into prokaryotic host cells are well-known in the art, and any suitable method can be used including but not limited to protoplast transformation, competent cell transformation, electroporation, conjugation, transduction, with DNA introduced as linearized or as circular polynucleotide. Persons skilled in the art will be readily able to identify a suitable method for introducing DNA into a given prokaryotic cell depending, e.g., on the genus. Methods for introducing DNA into prokaryotic host cells are for example described in Heinze et al., 2018, BMC Microbiology 18:56, Burke et al., 2001, Proc. Natl. Acad. Sci. USA 98: 6289-6294, Choi et al., 2006, J. Microbiol. Methods 64: 391-397, and Donald et al., 2013, J. Bacteriol. 195(11): 2612-2620.

In an aspect, the host cell is isolated, preferably the host cell is purified.

### Methods of Production

The present invention also relates to methods of producing a variant of the present invention, comprising (a) cultivating a recombinant host cell of the present invention under conditions conducive for production of the variant; and optionally (b) recovering the variant.

The host cell is cultivated in a nutrient medium suitable for production of the variant using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the variant to be expressed and/or isolated. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variant may be detected using methods known in the art that are specific for the variant, including, but not limited to, the use of specific antibodies, formation of an enzyme product, disappearance of an enzyme substrate, or an enzyme assay determining the relative or specific activity of the variant.

The variant may be recovered from the medium using methods known in the art, including, but not limited to, collection, centrifugation, filtration, extraction, spray-drying, evaporation, or precipitation. In one aspect, the whole fermentation broth is recovered. In another aspect, a cell-free fermentation broth comprising the polypeptide is recovered.

The variant may be purified by a variety of procedures known in the art to obtain substantially pure variants and/or fragments (see, e.g., Wingfield, 2015, Current Protocols in Protein Science; 80(1): 6.1.1-6.1.35; Labrou, 2014, Protein Downstream Processing, 1129: 3-10).

In an alternative aspect, the variant is not recovered.

### Mannanase Granules

The present invention also relates to enzyme granules/particles comprising a variant of the invention. In an embodiment, the granule comprises a core, and optionally one or more coatings (outer layers) surrounding the core.

The core may have a diameter, measured as equivalent spherical diameter (volume based average particle size), of 20-2000 μm, particularly 50-1500 μm, 100-1500 μm or 250-1200 μm. The core diameter, measured as equivalent spherical diameter, can be determined using laser diffraction, such as using a Malvern Mastersizer and/or the method described under ISO13320 (2020).

In an embodiment, the core comprises a variant of the present invention.

The core may include additional materials such as fillers, fiber materials (cellulose or synthetic fibers), stabilizing agents, solubilizing agents, suspension agents, viscosity regulating agents, light spheres, plasticizers, salts, lubricants and fragrances.

The core may include a binder, such as synthetic polymer, wax, fat, or carbohydrate.

The core may include a salt of a multivalent cation, a reducing agent, an antioxidant, a peroxide decomposing catalyst and/or an acidic buffer component, typically as a homogenous blend.

The core may include an inert particle with the variant absorbed into it, or applied onto the surface, e.g., by fluid bed coating.

The core may have a diameter of 20-2000 μm, particularly 50-1500 μm, 100-1500 μm or 250-1200 μm.

The core may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA).

The coating may be applied in an amount of at least 0.1% by weight of the core, e.g., at least 0.5%, at least 1%, at least 5%, at least 10%, or at least 15%. The amount may be at most 100%, 70%, 50%, 40% or 30%.

The coating is preferably at least 0.1 μm thick, particularly at least 0.5 μm, at least 1 μm or at least 5 μm. In some embodiments, the thickness of the coating is below 100 μm, such as below 60 μm, or below 40 μm.

The coating should encapsulate the core unit by forming a substantially continuous layer. A substantially continuous layer is to be understood as a coating having few or no holes, so that the core unit has few or no uncoated areas. The layer or coating should, in particular, be homogeneous in thickness.

The coating can further contain other materials as known in the art, e.g., fillers, antisticking agents, pigments, dyes, plasticizers and/or binders, such as titanium dioxide, kaolin, calcium carbonate or talc.

A salt coating may comprise at least 60% by weight of a salt, e.g., at least 65%, at least 70%, at least 75%, at least 80%, at least 85%, at least 90%, at least 95% or at least 99% by weight.

To provide acceptable protection, the salt coating is preferably at least 0.1 μm thick, e.g., at least 0.5 μm, at least 1 μm, at least 2 μm, at least 4 μm, at least 5 μm, or at least 8 μm. In a particular embodiment, the thickness of the salt coating is below 100 μm, such as below 60 μm, or below 40 μm.

The salt may be added from a salt solution where the salt is completely dissolved or from a salt suspension wherein the fine particles are less than 50 μm, such as less than 10 μm or less than 5 μm.

The salt coating may comprise a single salt or a mixture of two or more salts. The salt may be water soluble, in particular, having a solubility at least 0.1 g in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminum. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular, alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710.

Specific examples of suitable salts are NaCl (CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCl (CH_{20° C.}=85%), K₂HPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{25° C.}=86%). Other examples include NaH₂PO₄, (NH₄)H₂PO₄, CuSO₄, Mg(NO₃)₂ and magnesium acetate.

The salt may be in anhydrous form, or it may be a hydrated salt, i.e., a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄·7H₂O), zinc sulfate heptahydrate (ZnSO₄·7H₂O), sodium phosphate dibasic heptahydrate (Na₂HPO₄·7H₂O), magnesium nitrate hexahydrate (Mg(NO₃)₂(6H₂O)), sodium citrate dihydrate and magnesium acetate tetrahydrate.

Preferably the salt is applied as a solution of the salt, e.g., using a fluid bed.

The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The granule may optionally have one or more additional coatings. Examples of suitable coating materials are polyethylene glycol (PEG), methyl hydroxy-propyl cellulose (MHPC) and polyvinyl alcohol (PVA). Examples of enzyme granules with multiple coatings are described in WO 93/07263 and WO 97/23606.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in the Handbook of Powder Technology; Particle size enlargement by C. E. Capes; Vol. 1; 1980; Elsevier. Preparation methods include known feed and granule formulation technologies, e.g.,
(a) Spray dried products, wherein a liquid enzyme-containing solution is atomized in a spray drying tower to form small droplets which during their way down the drying tower dry to form an enzyme-containing particulate material. Very small particles can be produced this way (Michael S. Showell (editor); Powdered detergents; Surfactant Science Series; 1998; Vol. 71; pages 140-142; Marcel Dekker).
(b) Layered products, wherein the enzyme is coated as a layer around a pre-formed inert core particle, wherein an enzyme-containing solution is atomized, typically in a fluid bed apparatus wherein the pre-formed core particles are fluidized, and the enzyme-containing solution adheres to the core particles and dries up to leave a layer of dry enzyme on the surface of the core particle. Particles of a desired size can be obtained this way if a useful core particle of the desired size can be found. This type of product is described in, e.g., WO 97/23606.
(c) Absorbed core particles, wherein rather than coating the variant as a layer around the core, the enzyme is absorbed onto and/or into the surface of the core. Such a process is described in WO 97/39116.
(d) Extrusion or pelletized products, wherein a variant-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the enzyme paste, which is harmful to the enzyme (Michael S. Showell (editor); Powdered detergents; Surfactant Science Series; 1998; Vol. 71; pages 140-142; Marcel Dekker).
(e) Prilled products, wherein a variant-containing powder is suspended in molten wax and the suspension is sprayed, e.g., through a rotating disk atomizer, into a cooling chamber where the droplets quickly solidify (Michael S. Showell (editor); Powdered detergents; Surfactant Science Series; 1998; Vol. 71; pages 140-142; Marcel Dekker). The product obtained is one wherein the variant is uniformly distributed throughout an inert material instead of being concentrated on its surface. U.S. Pat. Nos. 4,016,040 and 4,713,245 describe this technique.
(f) Mixer granulation products, wherein a variant-containing liquid is added to a dry powder composition of conventional granulating components. The liquid and the powder in a suitable proportion are mixed and as the moisture of the liquid is absorbed in the dry powder, the components of the dry powder will start to adhere and agglomerate and particles will build up, forming granulates comprising the enzyme. Such a process is described in U.S. Pat. No. 4,106,991, EP 170360, EP 304332, EP 304331, WO 90/09440 and WO 90/09428. In a particular aspect of this process, various high-shear mixers can be used as granulators. Granulates consisting of variant, fillers and binders etc. are mixed with cellulose fibers to reinforce the particles to produce a so-called T-granulate. Reinforced particles, are more robust, and release less enzymatic dust.
(g) Size reduction, wherein the cores are produced by milling or crushing of larger particles, pellets, tablets, briquettes etc. containing the enzyme. The wanted core particle fraction is obtained by sieving the milled or crushed product. Over and undersized particles can be recycled. Size reduction is described in Martin Rhodes (editor); Principles of Powder Technology; 1990; Chapter 10; John Wiley & Sons.
(h) Fluid bed granulation. Fluid bed granulation involves suspending particulates in an air stream and spraying a liquid onto the fluidized particles via nozzles. Particles hit by spray droplets get wetted and become tacky. The tacky particles collide with other particles and adhere to them to form a granule.
(i) The cores may be subjected to drying, such as in a fluid bed drier. Other known methods for drying granules in the feed or enzyme industry can be used by the skilled person. The drying preferably takes place at a product temperature of from 25 to 90° C. For some enzymes, it is important the cores comprising the variant contain a low amount of water before coating with the salt. If water sensitive enzymes are coated with a salt before excessive water is removed, the excessive water will be trapped within the core and may affect the activity of the enzyme negatively. After drying, the cores preferably contain 0.1-10% w/w water.

Non-dusting granulates may be produced, e.g., as disclosed in U.S. Pat. Nos. 4,106,991 and 4,661,452 and may optionally be coated by methods known in the art.

The granulate may further comprise one or more additional enzymes. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D.

Another example of formulation of enzymes by the use of co-granulates is disclosed in WO 2013/188331.

The present invention also relates to protected enzymes prepared according to the method disclosed in EP 238216.

In an embodiment, the granule further comprises one or more additional enzymes selected from the group consisting of amylases, proteases, peroxidases, cellulases, betaglucanases, xyloglucanases, hemicellulases, xanthan endoglucanase, xanthan lyases, lipases, acyl transferases, phospholipases, esterases, laccases, catalases, aryl esterases, amylases, alpha-amylases, glucoamylases, cutinases, pectinases, pectate lyases, keratinases, reductases, oxidases, phenoloxidases, lipoxygenases, ligninases, carrageenases, pullulanases, tannases, arabinosidases, hyaluronidases, chondroitinases, xylanases, pectin acetyl esterases, polygalacturonases, rhamnogalacturonases, other endo-beta-mannanases, exo-beta-mannanases, pectin methylesterases, cellobiohydrolases, transglutaminases, licheninases, laminarinases, and DNAses, or any combination thereof.

### Liquid Formulations

The present invention also relates to liquid compositions comprising a variant of the invention. The composition may comprise an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

In some embodiments, filler(s) or carrier material(s) are included to increase the volume of such compositions. Suitable filler or carrier materials include, but are not limited to, various salts of sulfate, carbonate and silicate as well as talc, clay and the like. Suitable filler or carrier materials for liquid compositions include, but are not limited to, water or low molecular weight primary and secondary alcohols including polyols and diols. Examples of such alcohols include, but are not limited to, methanol, ethanol, propanol and isopropanol. In some embodiments, the compositions contain from about 5% to about 90% of such materials.

In an aspect, the liquid formulation comprises 20-80% w/w of polyol. In one embodiment, the liquid formulation comprises 0.001-2% w/w preservative.

In another embodiment, the invention relates to liquid formulations comprising:
(A) 0.001-25% w/w of a variant of the present invention;
(B) 20-80% w/w of polyol;
(C) optionally 0.001-2% w/w preservative; and
(D) water.

In another embodiment, the invention relates to liquid formulations comprising:
(A) 0.001-25% w/w of a variant of the present invention;
(B) 0.001-2% w/w preservative;
(C) optionally 20-80% w/w of polyol; and
(D) water.

In another embodiment, the liquid formulation comprises one or more formulating agents, such as a formulating agent selected from the group consisting of polyol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, PVA, acetate and phosphate, preferably selected from the group consisting of sodium sulfate, dextrin, cellulose, sodium thiosulfate, kaolin and calcium carbonate. In one embodiment, the polyols is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600, more preferably selected from the group consisting of glycerol, sorbitol and propylene glycol (MPG) or any combination thereof.

In another embodiment, the liquid formulation comprises 20-80% polyol (i.e., total amount of polyol), e.g., 25-75% polyol, 30-70% polyol, 35-65% polyol, or 40-60% polyol. In one embodiment, the liquid formulation comprises 20-80% polyol, e.g., 25-75% polyol, 30-70% polyol, 35-65% polyol, or 40-60% polyol, wherein the polyol is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600. In one embodiment, the liquid formulation comprises 20-80% polyol (i.e., total amount of polyol), e.g., 25-75% polyol, 30-70% polyol, 35-65% polyol, or 40-60% polyol, wherein the polyol is selected from the group consisting of glycerol, sorbitol and propylene glycol (MPG).

In another embodiment, the preservative is selected from the group consisting of sodium sorbate, potassium sorbate, sodium benzoate and potassium benzoate or any combination thereof. In one embodiment, the liquid formulation comprises 0.02-1.5% w/w preservative, e.g., 0.05-1% w/w preservative or 0.1-0.5% w/w preservative. In one embodiment, the liquid formulation comprises 0.001-2% w/w preservative (i.e., total amount of preservative), e.g., 0.02-1.5% w/w preservative, 0.05-1% w/w preservative, or 0.1-0.5% w/w preservative, wherein the preservative is selected from the group consisting of sodium sorbate, potassium sorbate, sodium benzoate and potassium benzoate or any combination thereof.

In another embodiment, the liquid formulation further comprises one or more additional enzymes selected from the group consisting of amylases, alpha-amylases, beta-amylases, beta-glucanases, proteases, oxidoreductase, peroxidases, cellulases, betaglucanases, xyloglucanases, hemicellulases, xanthan endoglucanases, xanthan lyases, lipases, acyl transferases, phospholipases, esterases, laccases, catalases, aryl esterases, amylases, alpha-amylases, glucoamylases, cutinases, pectinases, pectate lyases, keratinases, reductases, oxidases, phenoloxidases, lipoxygenases, ligninases, carrageenases, pullulanases, tannases, arabinosidases, hyaluronidases, chondroitinases, xylanases, pectin acetyl esterases, polygalacturonases, rhamnogalacturonases, other endo-beta-mannanases, exo-beta-mannanases, pectin methylesterases, cellobiohydrolases, transglutaminases, licheninases, laminarinases, and DNAses, or any combination thereof.

### Other Enzymes

In one embodiment, a mannanase variant of the invention is combined with one or more enzymes, such as at least two enzymes, more preferred at least three, four or five enzymes. Preferably, the enzymes have different substrate specificity, e.g., proteolytic activity, amylolytic activity, lipolytic activity, hemicellulytic activity, mannanase activity or pectolytic activity.

The detergent additive as well as the detergent composition may comprise one or more enzymes such as a protease, lipase, cutinase, an amylase, carbohydrase, cellulase, pectinase, additional mannanase, arabinase, galactanase, xylanase, oxidase, e.g., a laccase and/or peroxidase, a licheninase, laminarinase, a DNase.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

### Cellulases:

Suitable cellulases include those of animal, vegetable or microbial origin. Particularly suitable cellulases include those of bacterial or fungal origin. Chemically modified or protein engineered variants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases having color care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307 and WO 1999/001544.

Commercially available cellulases include Celluzyme™, and Carezyme™ (Novozymes A/S), Clazinase™, and Puradax HA™ (Genencor International Inc.), and KAC-500(B)™ (Kao Corporation).

### Proteases:

Suitable proteases include those of bacterial, fungal, plant, viral or animal origin e.g. microbial or vegetable origin. Microbial origin is preferred. Chemically modified or protein engineered variants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellulomonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens.

Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using the BPN′ numbering. More preferred the protease variants may comprise the mutations: S3T, V41, S9R, A15T, K27R, *36D, V68A, N76D, N87S,R, *97E, A98S, S99G,D,A, S99AD, S101G,M,R S103A, V104I,Y,N, S106A, G118V,R, H120D,N, N123S, S128L, P129Q, S130A, G160D, Y167A, R170S, A194P, G195E, V199M, V205I, L217D, N218D, M222S, A232V, K235L, Q236H, Q245R, N252K, T274A (using BPN′ numbering).

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes A/S), those sold under the tradename Maxatase®, Maxacal®, Maxapem®, Purafect®, Purafect Prime®, Preferenz™, Purafect MA®, Purafect Ox®, Purafect OxP®, Puramax®, Properase®, Effectenz™ FN2@, FN3@, FN4@, Excellase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

### Lipases:

Suitable lipases include those of animal, vegetable or microbial origin. Particularly suitable lipases include those of bacterial or fungal origin. Chemically modified or protein engineered variants are included. Examples of useful lipases include lipases from Humicola (synonym Thermomyces), e.g., from H. lanuginosa (T. lanuginosus) as described in EP 258 068 and EP 305 216 or from H. insolens as described in WO 96/13580, a Pseudomonas lipase, e.g., from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g., from B. subtilis (Dartois et al., 1993, Biochemica et Biophysica Acta, 1131: 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Preferred commercially available lipase enzymes include Lipolase™, Lipolase Ultra™, and Lipex™ (Novozymes A/S).

### Amylases:

Suitable amylases which can be used together with mannanase of the invention may be an alpha-amylase or a glucoamylase and may be of bacterial or fungal origin. Chemically modified or protein engineered variants are included. Amylases include, for example, alpha-amylases obtained from Bacillus, e.g., a special strain of Bacillus licheniformis, described in more detail in GB 1,296,839. Suitable amylases include amylases having SEQ ID NO: 3 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444. Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193. Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

$$\begin{matrix}M 197 T ; \\ H 156 Y + A 181 T + N 190 F + A 209 V + 02 \; 64 S ; or \\ G 48 A + T 491 + G 107 A + H 156 Y + A 181 T + N 190 F + I 201 F + A 209 V + 0264 S .\end{matrix}$$

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184. Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476. More preferred variants are those having a deletion in positions 181 and 182 or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476. Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264. Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:
N128C+K178L+T182G+Y305R+G475K;
N128C+K178L+T182G+F202Y+Y305R+D319T+G475K;
S125A+N128C+K178L+T182G+Y305R+G475K; or
S125A+N128C+T131I+T165|+K178L+T182G+Y305R+G475K wherein the variants are C-terminally truncated and optionally further comprises a substitution at position 243 and/or a deletion at position 180 and/or position 181. Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions. Other examples are amylase variants such as those described in WO2011/098531, WO2013/001078 and WO2013/001087. Commercially available amylases are Duramyl™, Termamyl™, Fungamyl™, Stainzyme™, Stainzyme Plus™, Natalase™ Liquozyme X and BAN™ (from Novozymes A/S), and Rapidase™, Purastar™/Effectenz™ Powerase and Preferenz S100 (from Genencor International Inc./DuPont).

### Peroxidases/Oxidases:

Suitable peroxidases/oxidases include those of plant, bacterial or fungal origin. Chemically modified or protein engineered variants are included. Examples of useful peroxidases include peroxidases from Coprinus, e.g., from C. cinereus, and variants thereof as those described in WO 93/24618, WO 95/10602, and WO 98/15257.

Commercially available peroxidases include Guardzyme™ (Novozymes A/S).

### Pectinases

Pectolytic enzymes (pectinases) can be classified according to their preferential substrate, highly methyl-esterified pectin or low methyl-esterified pectin and polygalacturonic acid (pectate), and their reaction mechanism, beta-elimination or hydrolysis. Pectinases can be mainly endo-acting, cutting the polymer at random sites within the chain to give a mixture of oligomers, or they may be exo-acting, attacking from one end of the polymer and producing monomers or dimers. Several pectinase activities acting on the smooth regions of pectin are included in the classification of enzymes provided by the Enzyme Nomenclature (1992) such as pectate lyase (EC 4.2.2.2), pectin lyase (EC 4.2.2.10), polygalacturonase (EC 3.2.1.15), exo-polygalacturonase (EC 3.2.1.67), exo-polygalacturonate lyase (EC 4.2.2.9) and exo-poly-alpha-galacturonosidase (EC 3.2.1.82).

Pectate lyases have been cloned from different bacterial genera such as Erwinia, Pseudomonas, Klebsiella and Xanthomonas. Also from Bacillus subtilis (Nasser et al. (1993) FEBS 335:319-326) and Bacillus sp. YA-14 (Kim et al. (1994) Biosci. Biotech. Biochem. 58:947-949) cloning of a pectate lyase has been described. The pectate lyases are generally characterised by an alkaline pH optimum and an absolute requirement for divalent cations, Ca²⁺ being the most stimulatory.

Variants of a pectate lyase from Bacillus subtilis have been disclosed inter alia in patent applications WO 2002/092741, WO 2003/095638 and WO 2018/007435.

### Xanthan Endoglucanase

Xanthan endoglucanases are endoglucanases exhibiting endo-beta-1,4-glucanase activity that is capable of catalysing hydrolysis of the 1,4-linked beta-D-glucose polymeric backbone of xanthan gum in conjunction with a suitable xanthan lyase enzyme. Such endoglucanases are disclosed in WO 2018/037062. Xanthan endoglucanase activity can be determined using the colorimetric assay developed by Lever (1972), Anal. Biochem. 47: 273-279, 1972.

An example of a xanthan endoglucanase is the polypeptide having SEQ ID NO: 31.

### Xanthan Lyase

Xanthan lyases are enzymes that cleave the beta-D-mannosyl-beta-D-1,4-glucuronosyl bond of xanthan and have been described in the literature. Xanthan lyases are known in the art, e.g. two xanthan lyases have been isolated from Paenibacillus alginolyticus XL-1 (e.g. Ruijssenaars et al. (1999) ‘A pyruvated mannose-specific xanthan lyase involved in xanthan degradation by Paenibacillus alginolyticus XL-1′, Appl. Environ. Microbiol. 65(6): 2446-2452, and Ruijssenaars et al. (2000),’ A novel gene encoding xanthan lyase of Paenibacillus alginolyticus strain XL-1′, Appl. Environ. Microbiol. 66(9): 3945-3950).

Xanthan lyase is classified according to the Enzyme Nomenclature as EC 4.2.2.12. This enzyme belongs to the family of lyases, specifically those carbon-oxygen lyases acting on polysaccharides.

WO 2017/046260 discloses polypeptides having xanthan degrading activity.

An example of a xanthan lyase is the polypeptide having SEQ ID NO: 30.

### Xyloglucanases

Xyloglucanses are capable of catalyzing the solubilization of xyloglucan to xyloglucan oligosaccharides. Some xyloglucanases only exhibit xyloglucanase activity, whereas others exhibit both xyloglucanase and cellulase activity. Xyloglucanses may be classified in EC 3.2.1.4 or EC. 3.2.1.151. Enzymes with xyloglucanase activity are for example described in Vincken et al. (1997) Carbohydrate Research 298(4):299-310, wherein three different endoglucanases EndoI, EndoV and EndoVI from Trichoderma viride (similar to T. reesei) are characterized. EndoI, EndoV and EndoVI belongs to family 5, 7 and 12 of glycosyl hydrolases, respectively, see Henrissat, B. (1991) Biochem. J. 280: 309-316, and Henrissat, B. and Bairoch, A. (1993) Biochem. J. 293: 781-788. WO 94/14953 discloses a family 12 xyloglucanase (EG II) cloned from the fungus Aspergillus aculeatus. WO 99/02663 discloses family 12 and family 5 xyloglucanases cloned from Bacillus licheniformis and Bacillus agaradhaerens, respectively. WO 01/062903 discloses family 44 xyloglucanases.

WO 2009/147210 discloses xyloglucanase variants. WO 99/02663 and WO 01/062903 suggest that xyloglucanases may be used in detergents.

### Lichenase/Beta-Glucanases:

Suitable lichenases (licheninases) include those of bacterial or fungal origin. They may be chemically modified, or protein engineered. Examples of useful beta-glucanases include those described in WO 2015/144824 (Novozymes A/S) and WO 99/06516 (Henkel KGAA).

### Nucleases:

Suitable nucleases include deoxyribonucleases (DNases) and ribonucleases (RNases) which are any enzyme that catalyzes the hydrolytic cleavage of phosphodiester linkages in the DNA or RNA backbone respectively, thus degrading DNA and RNA. There are two primary classifications based on the locus of activity. Exonucleases digest nucleic acids from the ends. Endonucleases act on regions in the middle of target molecules. The nuclease is preferably a DNase, which is preferable is obtainable from a microorganism, preferably a fungi or bacterium. In particular, a DNase which is obtainable from a species of Bacillus is preferred; in particular a DNase which is obtainable from Bacillus cibi, Bacillus subtilis or Bacillus licheniformis is preferred. Examples of such DNases are described in WO 2011/098579, WO2014/087011 and WO2017/060475. Particularly preferred is also a DNase obtainable from a species of Aspergillus; in particular a DNase which is obtainable from Aspergillus oryzae, such as a DNase described in WO 2015/155350.

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates as described above, liquids, in particular stabilized liquids, or slurries.

### Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S). Other commercially available mannanases are the mannanases disclosed in SEQ ID NO:8, SEQ ID NO: 19, SEQ ID NO: 20 and SEQ ID NO:21.

### Xanthanase

Complete enzymatic degradation of xanthan gum requires enzymatic activities including xanthan lyase activity and xanthan endoglucanase activity as described above. Xanthan lyases and endoglucanases for the degradation of xanthan gum and the use of such enzymes for cleaning purposes, such as the removal of xanthan gum containing stains, and in the drilling and oil industries are known in the art, e.g. from WO 2013/167581 A1.

In the context of the present invention the term Xanthanase (or xanthanase) means the combination of the enzymatic activities including xanthan lyase activity and xanthan endoglucanase activity.

A commercially available Xanthanase is the product Caledonia 100L from Novozymes A/S.

In a preferred aspect of the present invention the mannanase variants of the invention may be combined with at least one additional enzyme, such as at least two, three, four or five enzymes These additional enzymes are described in detail in the section “other enzymes”. Preferably, the enzymes have different substrate specificity, e.g., carbolytic activity, proteolytic activity, amylolytic activity, lipolytic activity, hemicellulytic activity, pectolytic activity or a mannanase with a different substrate specificity compared to the mannanase variants of the present invention, such as a GH5 mannanase. The enzyme combination may for example be a mannanase of the invention with another stain removing enzyme, e.g., a mannanase of the invention and a protease, such as a serine protease, a mannanase of the invention and an amylase, a mannanase of the invention and a cellulase, a mannanase of the invention and a lipase, a mannanase of the invention and a cutinase, a mannanase of the invention and a pectinase. More particularly preferred is a mannanase of the invention and another enzyme having carbolytic activity, e.g., a cellulase such as an endoglucanase and a beta-glucanase.

More preferably, the mannanase of the invention is combined with at least two other stain removing enzymes, e.g., a mannanase of the invention, a lipase and an amylase; or a mannanase of the invention, a protease and an amylase; or a mannanase of the invention, a protease and a lipase; or a mannanase of the invention, a protease and a pectinase; or a mannanase of the invention, a protease and a cellulase, such as an endoglucanase or a beta-glucanase; or a mannanase of the invention, a protease and a hemicellulase; or a mannanase of the invention, a protease and a cutinase; or a mannanase of the invention, an amylase and a pectinase; or a mannanase of the invention, an amylase and a cutinase; or a mannanase of the invention, an amylase and a cellulase such as an endoglucanase or a beta-glucanase; or a mannanase of the invention, an amylase and a hemicellulase; or a mannanase of the invention, a lipase and a pectinase; or a mannanase of the invention, a lipase and a cutinase; or a mannanase of the invention, a lipase and a cellulase such as an endoglucanase or a beta-glucanase; or a mannanase of the invention, a lipase and a hemicellulase. Even more preferably, a mannanase of the invention may be combined with at least three other stain removing enzymes, e.g., a mannanase of the invention, a protease, a lipase and an amylase; or a mannanase of the invention, a protease, an amylase and a pectinase; or a mannanase of the invention, a protease, an amylase and a cutinase; or a mannanase of the invention, a protease, an amylase and a cellulase; or a mannanase of the invention, a protease, an amylase and a hemicellulase; or a mannanase of the invention, an amylase, a lipase and a pectinase; or a mannanase of the invention, an amylase, a lipase and a cutinase; or a mannanase of the invention, an amylase, a lipase and a cellulase; or a mannanase of the invention, an amylase, a lipase and a hemicellulase; or a mannanase of the invention, a protease, a lipase and a pectinase; or a mannanase of the invention, a protease, a lipase and a cutinase; or a mannanase of the invention, a protease, a lipase and a cellulase; or a mannanase of the invention, a protease, a lipase and a hemicellulase. A mannanase according to the present invention may be combined with any of the enzymes selected from the non-exhaustive list comprising: carbohydrases, such as an amylase, a hemicellulase, a pectinase, a cellulase, a xanthan lyase activity, xanthan endoglucanase or a pullulanase, a peptidase, a protease or a lipase.

In a preferred embodiment, a mannanase of the invention is combined with a serine protease, e.g., an S8 family protease such as Savinase®.

In another embodiment of the present invention, a mannanase of the invention may be combined with one or more metalloproteases, such as an M4 metalloprotease, including Neutrase® or Thermolysin. Such combinations may further comprise combinations of the other detergent enzymes as outlined above.

In a particularly preferred embodiment the mannanase of the invention is combined with at GH5 mannanase, in particular a GH5 mannanase selected from the group of mannanases having at least 60%, such as at least 70% or at least 80% identity to the mannanase of any of SEQ ID NO: 8, SEQ ID NO: 18, SEQ ID NO: 19, SEQ ID NO: 20 or SEQ ID NO: 21, such as at least 85%, at least 90%, at least 95% or even 100% identity to the mannanase of any of SEQ ID NO: 8, SEQ ID NO: 18, SEQ ID NO: 19, SEQ ID NO: 20 or SEQ ID NO: 21.

In a more particular embodiment a mannanase variant having at least 80% identity to the mannanase of SEQ ID NO: 3, such as at least 85%, 90%, 95%, 96%, 97%, 98%, 99% or even 100% identity to the mannanase of SEQ ID NO: 3 is combined with a GH5 mannanase selected from the group of mannanases having at least 80% identity to the mannanase of any of SEQ ID NO: 8, SEQ ID NO: 18, SEQ ID NO: 19, SEQ ID NO: 20 or SEQ ID NO: 21, such as at least 85%, 90%, 95% or even 100% identity to the mannanase of any of SEQ ID NO: 8, SEQ ID NO: 18, SEQ ID NO: 19, SEQ ID NO: 20 or SEQ ID NO: 21.

In a further embodiment the mannanase variant of the invention is combined with a xanthan lyase or a xanthan endoglucanase or both a xanthan lyase and a xanthan endoglucanase. Preferred xanthan lyases have at least 80% identity to SEQ ID NO: 30, such as at least 85%, 90%, 95% or even 100% identity to the xanthan lyase of SEQ ID NO: 30. Preferred xanthan endoglucanases have at least 80% identity to SEQ ID NO: 31, such as at least 85%, at least 90%, at least 95% or even 100% identity to the xanthan endoglucanase of SEQ ID NO: 31.

In a more particular embodiment a mannanase variant having at least 80% identity to the mannanase of SEQ ID NO: 3, such as at least 85%, 90%, 95%, 96%, 97%, 98% or 99% identity to the mannanase of SEQ ID NO: 3 is combined with a xanthan lyase having at least 80% identity to SEQ ID NO: 30, such as at least 85%, 90%, 95% or even 100% identity to the xanthan lyase of SEQ ID NO: 30.

In another particular embodiment a mannanase variant having at least 80% identity to the mannanase of SEQ ID NO: 3, such as at least 85%, 90%, 95%, 96%, 97%, 98% or 99% identity to the mannanase of SEQ ID NO: 3 is combined with a xanthan endoglucanase having at least 80% identity to SEQ ID NO: 31, such as at least 85%, 90%, 95% or even 100% identity to the xanthan endoglucanase of SEQ ID NO: 31.

In a more particular embodiment a mannanase variant having at least 80% identity to the mannanase of SEQ ID NO: 3, such as at least 85%, 90%, 95%, 96%, 97%, 98% or 99% identity to the mannanase of SEQ ID NO: 3 is combined with a xanthan lyase having at least 80% identity to SEQ ID NO: 30, such as at least 85%, 90%, 95% or even 100% identity to the xanthan lyase of SEQ ID NO: 30 and with a xanthan endoglucanase having at least 80% identity to SEQ ID NO: 31, such as at least 85%, 90%, 95% or even 100% identity to the xanthan endoglucanase of SEQ ID NO: 31.

### Detergent Ingredients

### Surfactants

Typically, the detergent composition comprises (by weight of the composition) one or more surfactants in the range of 0% to 50%, preferably from 2% to 40%, more preferably from 5% to 35%, more preferably from 7% to 30%, most preferably from 10% to 25%, even most preferably from 15% to 20%. In a preferred embodiment the detergent is a liquid or powder detergent comprising less than 40%, preferably less than 30%, more preferably less than 25%, even more preferably less than 20% by weight of surfactant. The composition may comprise from 1% to 15%, preferably from 2% to 12%, 3% to 10%, most preferably from 4% to 8%, even most preferably from 4% to 6% of one or more surfactants. Preferred surfactants are anionic surfactants, non-ionic surfactants, cationic surfactants, zwitterionic surfactants, amphoteric surfactants, and mixtures thereof. Preferably, the major part of the surfactant is anionic. Suitable anionic surfactants are well known in the art and may comprise fatty acid carboxylates (soap), branched-chain, linear-chain and random chain alkyl sulfates or fatty alcohol sulfates or primary alcohol sulfates or alkyl benzenesulfonates such as LAS and LAB or phenylalknesulfonates or alkenyl sulfonates or alkenyl benzenesulfonates or alkyl ethoxysulfates or fatty alcohol ether sulfates or alpha-olefin sulfonate or dodecenyl/tetradecnylsuccinic acid. The anionic surfactants may be alkoxylated. The detergent composition may also comprise from 1 wt % to 10 wt % of non-ionic surfactant, preferably from 2 wt % to 8 wt %, more preferably from 3 wt % to 7 wt %, even more preferably less than 5 wt % of non-ionic surfactant. Suitable non-ionic surfactants are well known in the art and may comprise alcohol ethoxylates, and/or alkyl ethoxylates, and/or alkylphenol ethoxylates, and/or glucamides such as fatty acid N-glucosyl N-methyl amides, and/or alkyl polyglucosides and/or mono- or diethanolamides or fatty acid amides. The detergent composition may also comprise from 0 wt % to 10 wt % of cationic surfactant, preferably from 0.1 wt % to 8 wt %, more preferably from 0.5 wt % to 7 wt %, even more preferably less than 5 wt % of cationic surfactant. Suitable cationic surfactants are well known in the art and may comprise alkyl quaternary ammonium compounds, and/or alkyl pyridinium compounds and/or alkyl quaternary phosphonium compounds and/or alkyl ternary sulphonium compounds. The composition preferably comprises surfactant in an amount to provide from 100 ppm to 5,000 ppm surfactant in the wash liquor during the laundering process. The composition upon contact with water typically forms a wash liquor comprising from 0.5 g/I to 10 g/I detergent composition. Many suitable surface active compounds are available and fully described in the literature, for example, in “Surface-Active Agents and Detergents”, Volumes I and 11, by Schwartz, Perry and Berch. Also preferred are biobased surfactants, which may be wholly biobased (>95% biobased carbon of total carbon according to European standard EN 17035). As used herein biobased surfactants are a commercial or industrial product (other than food or feed) that is composed, in whole or in significant part, of biological products or renewable agricultural materials or forestry materials and/or as established by European standard EN 16575:2014. In particular rhamnolipids and sophorolipids may be used a detergent ingredient.

### Builders

The main role of builder is to sequester divalent metal ions (such as calcium and magnesium ions) from the wash solution that would otherwise interact negatively with the surfactant system. Builders are also effective at removing metal ions and inorganic soils from the fabric surface, leading to improved removal of particulate and beverage stains. Builders are also a source of alkalinity and buffer the pH of the wash water to a level of 9.5 to 11. The buffering capacity is also termed reserve alkalinity and should preferably be greater than 4.

The detergent compositions of the present invention may comprise one or more detergent builders or builder systems. Many suitable builder systems are described in the literature, for example in Powdered Detergents, Surfactant science series volume 71, Marcel Dekker, Inc. Builder may comprise from 0% to 60%, preferably from 5% to 45%, more preferably from 10% to 40%, most preferably from 15% to 35%, even more preferably from 20% to 30% builder by weight of the subject composition. The composition may comprise from 0% to 15%, preferably from 1% to 12%, 2% to 10%, most preferably from 3% to 8%, even most preferably from 4% to 6% of builder by weight of the subject composition.

Builders include, but are not limited to, the alkali metal, ammonium and alkanolammonium salts of polyphosphates (e.g., tripolyphosphate STPP), alkali metal silicates, alkaline earth and alkali metal carbonates, aluminosilicate builders (e.g., zeolite) and polycarboxylate compounds, ether hydroxypolycarboxylates, copolymers of maleic anhydride with ethylene or vinyl methyl ether, 1, 3, 5-trihydroxy benzene-2, 4, 6-trisulphonic acid, and carboxymethyloxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Ethanole amines (MEA, DEA, and TEA) may also contribute to the buffering capacity in liquid detergents.

### Bleaches

The detergent compositions of the present invention may comprise one or more bleaching agents. In particular powdered detergents may comprise one or more bleaching agents. Suitable bleaching agents include other photobleaches, pre-formed peracids, sources of hydrogen peroxide, bleach activators, hydrogen peroxide, bleach catalysts and mixtures thereof. In general, when a bleaching agent is used, the compositions of the present invention may comprise from about 0.1% to about 50% or even from about 0.1% to about 25% bleaching agent by weight of the subject cleaning composition. Examples of suitable bleaching agents include:
(1) other photobleaches for example Vitamin K3;
(2) preformed peracids: Suitable preformed peracids include, but are not limited to, compounds selected from the group consisting of percarboxylic acids and salts, percarbonic acids and salts, perimidic acids and salts, peroxymonosulfuric acids and salts, for example, Oxone, and mixtures thereof. Suitable percarboxylic acids include hydrophobic and hydrophilic peracids having the formula R—(C═O)O—O-M wherein R is an alkyl group, optionally branched, having, when the peracid is hydrophobic, from 6 to 14 carbon atoms, or from 8 to 12 carbon atoms and, when the peracid is hydrophilic, less than 6 carbon atoms or even less than 4 carbon atoms; and M is a counterion, for example, sodium, potassium or hydrogen;
(3) sources of hydrogen peroxide, for example, inorganic perhydrate salts, including alkali metal salts such as sodium salts of perborate (usually mono- or tetra-hydrate), percarbonate, persulphate, perphosphate, persilicate salts and mixtures thereof. In one aspect of the invention the inorganic perhydrate salts are selected from the group consisting of sodium salts of perborate, percarbonate and mixtures thereof. When employed, inorganic perhydrate salts are typically present in amounts of from 0.05 to 40 wt %, or 1 to 30 wt % of the overall composition and are typically incorporated into such compositions as a crystalline solid that may be coated. Suitable coatings include inorganic salts such as alkali metal silicate, carbonate or borate salts or mixtures thereof, or organic materials such as water-soluble or dispersible polymers, waxes, oils or fatty soaps. Useful bleaching compositions are described in U.S. Pat. Nos. 5,576,282, and 6,306,812;
(4) bleach activators having R—(C═O)-L wherein R is an alkyl group, optionally branched, having, when the bleach activator is hydrophobic, from 6 to 14 carbon atoms, or from 8 to 12 carbon atoms and, when the bleach activator is hydrophilic, less than 6 carbon atoms or even less than 4 carbon atoms; and L is leaving group. Examples of suitable leaving groups are benzoic acid and derivatives thereof—especially benzene sulphonate. Suitable bleach activators include dodecanoyl oxybenzene sulphonate, decanoyl oxybenzene sulphonate, decanoyl oxybenzoic acid or salts thereof, 3,5,5-trimethyl hexanoyloxybenzene sulphonate, tetraacetyl ethylene diamine (TAED) and nonanoyloxybenzene sulphonate (NOBS). Suitable bleach activators are also disclosed in WO 98/17767. While any suitable bleach activator may be employed, in one aspect of the invention the subject cleaning composition may comprise NOBS, TAED or mixtures thereof; and
(5) bleach catalysts that are capable of accepting an oxygen atom from peroxyacid and transferring the oxygen atom to an oxidizable substrate are described in WO 2008/007319. Suitable bleach catalysts include but are not limited to: iminium cations and polyions; iminium zwitterions; modified amines; modified amine oxides; N-sulphonyl imines; N-phosphonyl imines; N-acyl imines; thiadiazole dioxides; perfluoroimines; cyclic sugar ketones and mixtures thereof. The bleach catalyst will typically be comprised in the detergent composition at a level of from 0.0005% to 0.2%, from 0.001% to 0.1%, or even from 0.005% to 0.05% by weight.

When present, the peracid and/or bleach activator is generally present in the composition in an amount of from about 0.1 to about 60 wt %, from about 0.5 to about 40 wt % or even from about 0.6 to about 10 wt % based on the composition. One or more hydrophobic peracids or precursors thereof may be used in combination with one or more hydrophilic peracid or precursor thereof.

The amounts of hydrogen peroxide source and peracid or bleach activator may be selected such that the molar ratio of available oxygen (from the peroxide source) to peracid is from 1:1 to 35:1, or even 2:1 to 10:1.

### Adjunct Materials

Dispersants—The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms.

Dye Transfer Inhibiting Agents—The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent whitening agent—The detergent compositions of the present invention will preferably also contain additional components that may tint articles being cleaned, such as fluorescent whitening agent or optical brighteners. Any fluorescent whitening agent suitable for use in a laundry detergent composition may be used in the composition of the present invention. The most commonly used fluorescent whitening agents are those belonging to the classes of diaminostilbene-sulphonic acid derivatives, diarylpyrazoline derivatives and bisphenyl-distyryl derivatives.

Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4 anilino-s-triazin-6-ylamino) stilbene disulphonate. Tinopal CBS is the disodium salt of 2,2′-bis-(phenyl-styryl) disulphonate.

Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

Fabric hueing agents—The detergent compositions of the present invention may also include fabric hueing agents such as dyes or pigments which when formulated in detergent compositions can deposit onto a fabric when said fabric is contacted with a wash liquor comprising said detergent compositions thus altering the tint of said fabric through absorption of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 2005/03274, WO 2005/03275, WO 2005/03276 and EP 1 876 226. The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch.

Soil release polymers—The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series, volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523. Furthermore random graft co-polymers are suitable soil release polymers Suitable graft co-polymers are described in more detail in WO 2007/138054, WO 2006/108856 and WO 2006/113314. Other soil release polymers are substituted polysaccharide structures especially substituted cellulosic structures such as modified cellulose deriviatives such as those described in EP 1 867 808 or WO 2003/040279. Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-redeposition agents—The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, structurants for liquid detergents and/or structure elasticizing agents.

In one aspect the detergent is a compact fluid laundry detergent composition comprising: a) at least about 10%, preferably from 20 to 80% by weight of the composition, of surfactant selected from anionic surfactants, non ionic surfactants, soap and mixtures thereof; b) from about 1% to about 30%, preferably from 5 to 30%, by weight of the composition, of water; c) from about 1% to about 15%, preferably from 3 to 10% by weight of the composition, of non-aminofunctional solvent; and d) from about 5% to about 20%, by weight of the composition, of a performance additive selected from chelants, soil release polymers, enzymes and mixtures thereof; wherein the compact fluid laundry detergent composition comprises at least one of: (i) the surfactant has a weight ratio of the anionic surfactant to the nonionic surfactant from about 1.5:1 to about 5:1, the surfactant comprises from about 15% to about 40%, by weight of the composition, of anionic surfactant and comprises from about 5% to about 40%, by weight of the composition, of the soap; (ii) from about 0.1% to about 10%, by weight of the composition, of a suds boosting agent selected from suds boosting polymers, cationic surfactants, zwitterionic surfactants, amine oxide surfactants, amphoteric surfactants, and mixtures thereof; and (ii) both (i) and (ii). All the ingredients are described in WO 2007/130562. Further polymers useful in detergent formulations are described in WO 2007/149806.

In another aspect the detergent is a compact granular (powdered) detergent comprising a) at least about 10%, preferably from 15 to 60% by weight of the composition, of surfactant selected from anionic surfactants, non-ionic surfactants, soap and mixtures thereof; b) from about 10 to 80% by weight of the composition, of a builder, preferably from 20% to 60% where the builder may be a mixture of builders selected from i) phosphate builder, preferably less than 20%, more preferably less than 10% even more preferably less than 5% of the total builder is a phosphate builder; ii) a zeolite builder, preferably less than 20%, more preferably less than 10% even more preferably less than 5% of the total builder is a zeolite builder; iii) citrate, preferably 0 to 5% of the total builder is a citrate builder; iv) polycarboxylate, preferably 0 to 5% of the total builder is a polycarboxylate builder v) carbonate, preferably 0 to 30% of the total builder is a carbonate builder and vi) sodium silicates, preferably 0 to 20% of the total builder is a sodium silicate builder; c) from about 0% to 25% by weight of the composition, of fillers such as sulphate salts, preferably from 1% to 15%, more preferably from 2% to 10%, more preferably from 3% to 5% by weight of the composition, of fillers; and d) from about 0.1% to 20% by weight of the composition, of enzymes, preferably from 1% to 15%, more preferably from 2% to 10% by weight of the composition, of enzymes.

The soils and stains that are important for detergent formulators are composed of many different substances, and a range of different enzymes, all with different substrate specificities have been developed for use in detergents both in relation to laundry and hard surface cleaning, such as dishwashing. These enzymes are considered to provide an enzyme detergency benefit, since they specifically improve stain removal in the cleaning process they are applied in as compared to the same process without enzymes. Stain removing enzymes that are known in the art include enzymes such as carbohydrases, amylases, proteases, lipases, cellulases, hemicellulases, xylanases, cutinases, and pectinase.

The cleaning process or the textile care process may for example be a laundry process, a dishwashing process or cleaning of hard surfaces such as bathroom tiles, floors, tabletops, drains, sinks and washbasins. Laundry processes can for example be household laundering, but it may also be industrial laundering. Furthermore, the invention relates to a process for laundering of fabrics and/or garments where the process comprises treating fabrics with a washing solution containing a detergent composition, and at least one mannanase of the invention. The cleaning process or a textile care process can for example be carried out in a machine-washing process or in a manual washing process. The washing solution can for example be an aqueous washing solution containing a detergent composition.

The fabrics and/or garments subjected to a washing, cleaning or textile care process of the present invention may be conventional washable laundry, for example household laundry. Preferably, the major part of the laundry is garments and fabrics, including knits, woven, denims, non-woven, felts, yarns, and towelling. The fabrics may be cellulose based such as natural cellulosics, including cotton, flax, linen, jute, ramie, sisal or coir or manmade cellulosics (e.g., originating from wood pulp) including viscose/rayon, ramie, cellulose acetate fibers (tricell), lyocell or blends thereof. The fabrics may also be non-cellulose based such as natural polyamides including wool, camel, cashmere, mohair, rabit and silk or synthetic polymer such as nylon, aramid, polyester, acrylic, polypropylen and spandex/elastane, or blends thereof as well as blend of cellulose based and non-cellulose based fibers. Examples of blends are blends of cotton and/or rayon/viscose with one or more companion material such as wool, synthetic fibers (e.g., polyamide fibers, acrylic fibers, polyester fibers, polyvinyl alcohol fibers, polyvinyl chloride fibers, polyurethane fibers, polyurea fibers, aramid fibers), and cellulose-containing fibers (e.g., rayon/viscose, ramie, flax, linen, jute, cellulose acetate fibers, lyocell).

The last few years there has been an increasing interest in replacing components in detergents, which is derived from petrochemicals with renewable biological components such as enzymes and polypeptides without compromising the wash performance. When the components of detergent compositions change new enzyme activities or new enzymes having alternative and/or improved properties compared to the commonly used detergent enzymes such as proteases, lipases and amylases is needed to achieve a similar or improved wash performance when compared to the traditional detergent compositions.

Typical detergent compositions include various components in addition to the enzymes, these components have different effects, some components like the surfactants lower the surface tension in the detergent, which allows the stain being cleaned to be lifted and dispersed and then washed away, other components like bleach systems removes discolor often by oxidation and many bleaches also have strong bactericidal properties and are used for disinfecting and sterilizing. Yet other components like builder and chelator softens, e.g., the wash water by removing the metal ions from the liquid.

In a particular embodiment, the invention concerns the use of a composition comprising a mannanase of the invention, wherein said composition further comprises at least one or more of the following a surfactant, a builder, a chelator or chelating agent, bleach system or bleach component in laundry or dish wash.

In a preferred embodiment of the invention the amount of a surfactant, a builder, a chelator or chelating agent, bleach system and/or bleach component are reduced compared to amount of surfactant, builder, chelator or chelating agent, bleach system and/or bleach component used without the added mannanase of the invention. Preferably the at least one component which is a surfactant, a builder, a chelator or chelating agent, bleach system and/or bleach component is present in an amount that is 1% less, such as 2% less, such as 3% less, such as 4% less, such as 5% less, such as 6% less, such as 7% less, such as 8% less, such as 9% less, such as 10% less, such as 15% less, such as 20% less, such as 25% less, such as 30% less, such as 35% less, such as 40% less, such as 45% less, such as 50% less than the amount of the component in the system without the addition of mannanase of the invention, such as a conventional amount of such component. In one aspect, the mannanase of the invention is used in detergent compositions wherein said composition is free of at least one component which is a surfactant, a builder, a chelator or chelating agent, bleach system or bleach component and/or polymer.

Also preferred are biobased surfactants, which may be wholly biobased (>95% biobased carbon of total carbon according to European standard EN 17035). As used herein biobased surfactants are a commercial or industrial product (other than food or feed) that is composed, in whole or in significant part, of biological products or renewable agricultural materials or forestry materials and/or as established by European standard EN 16575:2014.

Additional bio-based surfactants may be used e.g. wherein the surfactant is a sugar-based non-ionic surfactant which may be a hexyl-β-D-maltopyranoside, thiomaltopyranoside or a cyclic-maltopyranoside, such as described in EP2516606 B1. Other biobased surfactants may include rhamnolipids and sophorolipids.

When included in the detergent, biobased surfactants are in amounts of 1-25% (and/or lower levels than the above-designated surfactants).

In an embodiment is provided a detergent composition comprising a mannanase variant as described herein and an additional mannanase, and wherein the additional mannanase is a GH5 mannanase, such as a mannanase having at least 50%, such as 60%, 70%, 80%, 90%, 95% or even 100% identity to any of SEQ ID NO:8, SEQ ID NO: 18, SEQ ID NO: 19, SEQ ID: 20 or SEQ ID NO: 21. Preferably the ratio between mannanase variant of the present invention and GH5 mannanase is in the range 1:3 to 3:1.

### Uses

The mannanases variants of the invention may be used in applications where mannan needs to be degraded. Examples of where mannanases could be used are in the production of bioethanol from softwood and palm kernel press cake, for the improvement of animal feed and in the hydrolysis of coffee. Furthermore, guar gum is used in many food products and in the oil and gas industry, so the mannanases of the invention could be used in detergents to remove mannan containing stains, for hydraulic fracturing to create subterranean fractures that extend from the borehole into rock formation in order to increase the rate at which fluids can be produced by the formation or for cleaning borehole filtercake. The mannan may thus be used in fracturing of a subterranean formation perpetrated by a well bore or the mannan may be used as a component in borehole filtercake.

In one aspect, the mannanase variants of aspect one or two, detergent composition of aspect three or four, granule of aspect five or six or liquid formulation of aspect seven or eight may be used for degrading mannan, such as linear mannan, galactomannan, glucomannan and galactoglucomannan. In one aspect, the mannanase variants of aspect one or two, detergent composition of aspect three or four, granule of aspect five or six or liquid formulation of aspect seven or eight may be used in a process for degrading mannan, such as linear mannan, galactomannan, glucomannan and galactoglucomannan.

In one aspect, the mannanase variants of aspect one or two, detergent composition of aspect three or four, granule of aspect five or six or liquid formulation of aspect seven or eight may be used for controlling the viscosity of drilling fluids. In one aspect, the mannanase variants of aspect one or two, detergent composition of aspect three or four, granule of aspect five or six or liquid formulation of aspect seven or eight may be used in fracturing of a subterranean formation perpetrated by a well bore.

The mannanases variants of the invention may be used for preventing, reducing or removing malodor from an item. Thus, in one embodiment, the mannanase variants of aspect one or two, detergent composition of aspect three or four, granule of aspect five or six or liquid formulation of aspect seven or eight may be used for preventing, reducing or removing malodor from an item.

### Washing Method

The detergent compositions of the present invention are ideally suited for use in laundry applications. Accordingly, the present invention includes a method for laundering a fabric. The method comprises the steps of contacting a fabric to be laundered with a cleaning laundry solution comprising the detergent composition according to the invention. The fabric may comprise any fabric capable of being laundered in normal consumer use conditions. The solution preferably has a pH of from about 5.5 to about 8. The compositions may be employed at concentrations of from about 100 ppm, preferably 500 ppm to about 15,000 ppm in solution. The water temperatures typically range from about 5° C. to about 90° C., including about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C. and about 90° C. The water to fabric ratio is typically from about 1:1 to about 30:1.

In particular embodiments, the washing method is conducted at a pH of from about 5.0 to about 11.5, or in alternative embodiments, even from about 6 to about 10.5, such as about 5 to about 11, about 5 to about 10, about 5 to about 9, about 5 to about 8, about 5 to about 7, about 5.5 to about 11, about 5.5 to about 10, about 5.5 to about 9, about 5.5 to about 8, about 5.5. to about 7, about 6 to about 11, about 6 to about 10, about 6 to about 9, about 6 to about 8, about 6 to about 7, about 6.5 to about 11, about 6.5 to about 10, about 6.5 to about 9, about 6.5 to about 8, about 6.5 to about 7, about 7 to about 11, about 7 to about 10, about 7 to about 9, or about 7 to about 8, preferably about 5.5 to about 9, and more preferably about 6 to about 8.

In particular embodiments, the washing method is conducted at a degree of hardness of from about 0° dH to about 30° dH, such as about 1° dH, about 2°dH, about 3°dH, about 4°dH, about 5°dH, about 6°dH, about 7°dH, about 8°dH, about 9°dH, about 10° dH, about 11° dH, about 12°dH, about 13°dH, about 14°dH, about 15°dH, about 16°dH, about 17°dH, about 18°dH, about 19°dH, about 20°dH, about 21° dH, about 22°dH, about 23°dH, about 24°dH, about 25°dH, about 26°dH, about 27°dH, about 28°dH, about 29° dH, about 30°dH. Under typical European wash conditions, the degree of hardness is about 15°dH, under typical US wash conditions about 6°dH, and under typical Asian wash conditions, about 3°dH.

The present invention relates to a method of cleaning a fabric, a dishware or hard surface with a detergent composition comprising a mannanase variants of the invention.

A preferred embodiment concerns a method of cleaning, said method comprising the steps of: contacting an object with a cleaning composition comprising a mannanase variants of the invention under conditions suitable for cleaning said object. In a preferred embodiment the cleaning composition is a detergent composition and the process is a laundry or a dish wash process.

Still another embodiment relates to a method for removing stains from fabric which comprises contacting said a fabric with a composition comprising a mannanase variants of the invention under conditions suitable for cleaning said object.

### Low Temperature Uses

One embodiment of the invention concerns a method of doing laundry, dish wash or industrial cleaning comprising contacting a surface to be cleaned with a mannanase variants of the invention, and wherein said laundry, dish wash, industrial or institutional cleaning is performed at a temperature of about 40° C. or below. One embodiment of the invention relates to the use of a mannanase in laundry, dish wash or a cleaning process wherein the temperature in laundry, dish wash, industrial cleaning is about 40° C. or below

In another embodiment, the invention concerns the use of a mannanase according to the invention in a protein removing process, wherein the temperature in the protein removing process is about 40° C. or below.

In each of the above-identified methods and uses, the wash temperature is about 40° C. or below, such as about 39° C. or below, such as about 38° C. or below, such as about 37° C. or below, such as about 36° C. or below, such as about 35° C. or below, such as about 34° C. or below, such as about 33° C. or below, such as about 32° C. or below, such as about 31° C. or below, such as about 30° C. or below, such as about 29° C. or below, such as about 28° C. or below, such as about 27° C. or below, such as about 26° C. or below, such as about 25° C. or below, such as about 24° C. or below, such as about 23° C. or below, such as about 22° C. or below, such as about 21° C. or below, such as about 20° C. or below, such as about 19° C. or below, such as about 18° C. or below, such as about 17° C. or below, such as about 16° C. or below, such as about 15° C. or below, such as about 14° C. or below, such as about 13° C. or below, such as about 12° C. or below, such as about 11° C. or below, such as about 10° C. or below, such as about 9° C. or below, such as about 8° C. or below, such as about 7° C. or below, such as about 6° C. or below, such as about 5° C. or below, such as about 40C or below, such as about 3° C. or below, such as about 2° C. or below, such as about 1° C. or below.

In another preferred embodiment, the wash temperature is in the range of about 5-40° C., such as about 5-30° C., about 5-20° C., about 5-10° C., about 10-40° C., about 10-30° C., about 10-20° C., about 15-40° C., about 15-30° C., about 15-20° C., about 20-40° C., about 20-30° C., about 25-40° C., about 25-30° C., or about 30-40° C. In particular preferred embodiments the wash temperature is about 20° C., about 30° C., or about 40° C.

### Use of Mannanases of the Invention in Preventing, Reducing or Removing a Biofilm

Biofilm can develop on textile when microorganisms are present on an item and stick together on the item. Some microorganisms tend to adhere to the surface of items such as textiles. Some microorganisms adhere to such surfaces and form a biofilm on the surface. The biofilm may be sticky and the adhered microorganisms and/or the biofilm may be difficult to remove. Furthermore, the biofilm adhere soil due to the sticky nature of the biofilm. The commercial laundry detergent compositions available on the marked do not remove such adhered microorganisms or biofilm.

### Use of Mannanases of the Invention in Food Processing and Animal Feed

Several anti-nutritional factors can limit the use of specific plant material in the preparation of animal feed and food for humans. For example, plant material containing oligomannans such as mannan, galactomannan, glucomannan and galactoglucomannan can reduce the digestibility and absorption of nutritional compounds such as minerals, vitamins, sugars and fats by the animals. The negative effects are in particular due to the high viscosity of the mannan-containing polymers and to the ability of the mannan-containing polymers to adsorb nutritional compounds. These effects are reduced using mannan-containing polymers degrading enzymes, namely endo-beta-mannanase enzymes such as the mannanases described herein, which permit a higher proportion of mannan-containing polymers containing cheap plant material to be included in the feed resulting in a reduction of feed costs. Additionally, through the activity of the mannanases of the invention, mannan-containing polymers are broken down to simpler sugars, which can be more readily assimilated to provide additional energy. Accordingly, the invention further relates to using the mannanases of the invention for processing and/or manufacturing of food or animal feed.

Accordingly, the present invention relates to an animal feed composition and/or animal feed additive composition and/or pet food comprising a mannanase variants of the invention.

The present invention further relates to a method for preparing such animal feed composition and/or animal feed additive composition and/or pet food comprising mixing the mannanase variants of the invention with one or more animal feed ingredients and/or animal feed additive ingredients and/or pet food ingredients.

Furthermore, the present invention relates to the use of the mannanase variants of the invention in the preparation of an animal feed composition and/or animal feed additive composition and/or pet food.

### Use of Mannanases of the Invention for Fermented Beverages

In one aspect, the invention relates to a method of preparing a fermented beverage, such as beer or wine, comprising mixing the mannanase variants of aspect one or two, granule of aspect five or six or liquid formulation of aspect seven or eight with malt and/or adjunct.

Another aspect concerns a method of providing a fermented beverage comprising the step of contacting a mash and/or a wort with the mannanase variants of aspect one or two, the granule of aspect five or six or the liquid formulation of aspect seven or eight.

In the context of the present invention, the term “fermented beverage” is meant to comprise any beverage such as wine or beer produced by a method comprising a fermentation process, such as a microbial, bacterial and/or yeast fermentation.

In an aspect of the invention the fermented beverage is beer. The term “beer” is meant to comprise any fermented wort produced by fermentation/brewing of a starch-containing plant material. Often, beer is produced from malt or adjunct, or any combination of malt and adjunct as the starch-containing plant material. As used herein the term “malt” is understood as any malted cereal grain, such as malted barley or wheat.

As used herein the term “adjunct” refers to any starch and/or sugar containing plant material which is not malt, such as barley or wheat malt. As examples of adjuncts, mention can be made of materials such as common corn grits, refined corn grits, brewer's milled yeast, rice, sorghum, refined corn starch, barley, barley starch, dehusked barley, wheat, wheat starch, torrified cereal, cereal flakes, rye, oats, potato, tapioca, cassava and syrups, such as corn syrup, sugar cane syrup, inverted sugar syrup, barley and/or wheat syrups, and the like may be used as a source of starch

As used herein, the term “mash” refers to an aqueous slurry of any starch and/or sugar containing plant material such as grist, e. g. comprising crushed barley malt, crushed barley, and/or other adjunct or a combination hereof, mixed with water later to be separated into wort and spent grains.

As used herein, the term “wort” refers to the unfermented liquor run-off following extracting the grist during mashing.

### Use of Mannanases of the Invention for Treating Coffee Extracts

The mannanase variants of the invention may also be used for hydrolyzing galactomannans present in liquid coffee extracts. In certain preferred embodiments, the mannanase variants of the invention is used to inhibit gel formation during freeze drying of liquid coffee extracts. The decreased viscosity of the extract reduces the energy consumption during drying. In certain other preferred embodiments, the mannanase variants of the invention is applied in an immobilized form in order to reduce enzyme consumption and avoid contamination of the coffee extract. This use is further disclosed in EP 676 145.

In general terms the coffee extract is incubated in the presence of an isolated mannanase variants of the invention or fragment or variant thereof under conditions suitable for hydrolyzing galactomannans present in liquid coffee extract.

Thus, in one embodiment, then invention relates to a process for producing a coffee extract, comprising the steps:
(a) providing roast and ground coffee beans;
(b) adding to said coffee beans water and the polypeptide of aspect one or two, granule of aspect five or six or liquid formulation of aspect seven or eight;
(c) incubating to make an aqueous coffee extract; and
(d) separating the coffee extract from the extracted coffee beans.

### Use of Mannanase of the Invention in Bakery Food Products

In another aspect, the invention relates to a method of preparing baked products comprising adding the mannanase variants of aspect one or two, granule of aspect five or six or liquid formulation of aspect seven or eight to a dough, followed by baking the dough.

Examples of baked products are well known to those skilled in the art and include breads, rolls, puff pastries, sweet fermented doughs, buns, cakes, crackers, cookies, biscuits, waffles, wafers, tortillas, breakfast cereals, extruded products, and the like.

The mannanase variants of the invention may be added to dough as part of a bread improver composition. Bread improvers are compositions containing a variety of ingredients, which improve dough properties and the quality of bakery products, e.g. bread and cakes. Bread improvers are often added in industrial bakery processes because of their beneficial effects e.g. the dough stability and the bread texture and volume. Bread improvers usually contain fats and oils as well as additives like emulsifiers, enzymes, antioxidants, oxidants, stabilizers and reducing agents. In addition to the mannanase variants of the invention, other enzymes which may also be present in the bread improver including amylases, hemicellulases, amylolytic complexes, lipases, proteases, xylanases, pectinases, pullulanases, non-starch polysaccharide degrading enzymes and redox enzymes like glucose oxidase, lipoxygenase or ascorbic acid oxidase.

In one aspect, the mannanase variants of the invention may be added to dough as part of a bread improver composition which also comprises a glucomannan and/or galactomannan source such as konjac gum, guar gum, locust bean gum (Ceratonia siliqua), copra meal, ivory nut mannan (Phyteleohas macrocarpa), seaweed mannan extract, coconut meal, and the cell wall of brewers yeast (may be dried, or used in the form of brewers yeast extract).

A further aspect of the invention relates to the use of the mannanase variants of the invention in dough to improve dough tolerance, flexibility and stickiness. Preferably the dough to which the mannanase variants of the invention may be added is not a pure wheat flour dough, but comprises bran or oat, rice, millet, maize, or legume flour in addition to or instead of pure wheat flour.

A yet further aspect of the invention relates to the use of any of the mannanase variants of the invention in dough to improve the crumb structure and retard staling in the final baked product, such as bread.

### Use of Mannanase of the Invention for Use in Dairy Food Products

In one aspect of the current invention, the mannanase variants of the invention may be added to milk or any other dairy product to which has also been added a glucomannan and/or galactomannan. Typical glucomannan and/or galactomannan sources are listed above in the bakery aspects, and include guar or konjac gum. The combination of the mannanase variants of the invention with a glucomannan and/or galactomannan releases mannanase hydrolysates (mannooligosaccharides) which act as soluble prebiotics by promoting the selective growth and proliferation of probiotic bacteria (especially Bifidobacteria and Lactobacillus lactic acid bacteria) commonly associated with good health when found at favourable population densities in the large intestine or colon.

In one aspect, the invention relates to a method of preparing milk or dairy products comprising adding to the milk or dairy product (a) glucomannan, galactomannan and/or galactoglucomannan and (b) the mannanase variants of aspect one or two, granule of aspect five or six or liquid formulation of aspect seven or eight.

In one aspect of the invention, the mannanase variants of the invention is used in combination with any glucomannan or galactomannan prior to or following addition to a dairy based foodstuff to produce a dairy based foodstuff comprising prebiotic mannan hydrolysates. In a further aspect of the invention the thus produced mannooligosacharide-containing dairy product is capable of increasing the population of beneficial human intestinal microflora, and in a yet further aspect of the current invention the dairy based foodstuff may comprise the mannanase variants of the invention together with any source of glucomannan and/or galactomannan and/or galactoglucomannan, and a dose sufficient for inoculation of at least one strain of bacteria (such as Bifidobacteria or Lactobacillus) known to be of benefit in the human large intestine. Preferably said dairy-based foodstuff is a yoghurt or milk drink.

### Use of Mannanase of the Invention for Paper Pulp Bleaching

The mannanase variants of the invention may further be used in the enzyme aided bleaching of paper pulps such as chemical pulps, semi-chemical pulps, kraft pulps, mechanical pulps or pulps prepared by the sulfite method. Thus, the invention relates to a method of bleaching paper pulps comprising incubating the paper pulp with the polypeptide of aspect one or two, detergent composition of aspect three or four, granule of aspect five or six or liquid formulation of aspect seven or eight.

In some embodiments, the pulps are chlorine free pulps bleached with oxygen, ozone, peroxide or peroxyacids. In some embodiments, the mannanase variants of the invention is used in enzyme aided bleaching of pulps produced by modified or continuous pulping methods that exhibit low lignin contents. In some other embodiments, the mannanase variants of the invention is applied alone or preferably in combination with xylanase and/or endoglucanase and/or alpha-galactosidase and/or cellobiohydrolase enzymes.

### EMBODIMENTS

The invention is further defined by the following numbered paragraphs:
1) A variant of the polypeptide of SEQ ID NO: 2 wherein the amino acids at position 490 and 491 are deleted, wherein the variant has at least 60% sequence identity to the polypeptide of SEQ ID NO: 3 and wherein the variant has mannanase activity.
2) The variant according to embodiment 1 wherein the variant comprises an extension of one or more amino acids at the N-terminal and/or C-terminal ends or a truncation of one or more amino acids at the N-terminal and/or C-terminal ends with the proviso that an insertion at the C-terminal is not *490W and *491R
3) The variant according to embodiment 1 or 2 comprising deletion of at least 11 amino acids from the N-terminal end, such as amino acid 1 to 12, such as amino acid 1 to 13, such as amino acid 1 to 14 or such as amino acid 1 to 15 of SEQ ID NO: 2.
4) The variant according to any of embodiments 1 to 3 comprising the deletions A1*+12*+G3*+V4*+P5*+G6*+G7*+V8*+A9*+E10*+P11*+H12*+T13*+S14*+Q15*.
5) The variant of any of embodiments 1 to 4 further comprising the substitution D16A.
6) The variant according to any of embodiments 1 to 5 further comprising one or more modifications selected from the group consisting of I30L, D48P, Y155H, T167P, Q215E, H276C, R280K, F286C, G366N, and D486E.
7) The variant according to embodiment 6 further comprising one or more modifications selected from the group consisting of G20P, A101L, A111P, A118E, S137A, Q143R, R160L, V161A, E162D, R164S, R164P, R164A, R164K, I165G, I165A, I165P, I165T, I165E, M171I, N176S, P182R, Q183E, V244A, H324K, and D385H.
8) The variant according to any of embodiments 1 to 7, wherein the variant has at least 65%, 70%, 75%, 80%, 85%, 85.5%, 86%, 86.5%, 87%, 87.5%, 88%, 88.5%, 89%, 89.5%, 90%, 90.5%, such as at least 91%, at least 91.5%, at least 92, at least 92.5%, at least 93%, at least 93.5%, at least 93.9%, at least 94%, at least 94.5%, at least 95%, at least 95.5%, at least 96%, at least 96.5%, at least 97%, at least 97.5%, at least 98%, at least 98.5%, at least 99%, at least 99.5%, or 99.9% or even 100% sequence identity to the polypeptide of SEQ ID NO: 3, the polypeptide of SEQ ID NO: 4, the polypeptide of SEQ ID NO: 5, the polypeptide of SEQ ID NO:6 or the polypeptide of SEQ ID NO: 7, the polypeptide of SEQ ID NO: 22, the polypeptide of SEQ ID NO: 23, the polypeptide of SEQ ID NO: 24, the polypeptide of SEQ ID NO: 25, the polypeptide of SEQ ID NO: 26, the polypeptide of SEQ ID NO: 27, the polypeptide of SEQ ID NO: 28, or the polypeptide of SEQ ID NO: 29, wherein the variant has mannanase activity.
9) An enzyme composition comprising a variant of any of claims 1 to 8 and a GH5 mannanase, wherein the enzyme composition is a granulated product or a liquid product, wherein the ratio between the GH5 mannanase and the variant of any of embodiments 1 to 9 is in the ratio from 1:3 to 3:1 based on weight of enzyme protein.
10) A detergent composition comprising the variant of any of embodiments 1 to 8 and at least one detergent adjunct ingredient.
11) The detergent composition according to embodiment 10 further comprising the enzyme composition of embodiment 9.
12) The detergent composition according to embodiment embodiment 11, wherein the GH5 mannanase has at least 60%, 70% or 80% identity to the mannanase of any of SEQ ID NO: 8, SEQ ID NO: 18, SEQ ID NO: 19, SEQ ID NO: 20 or SEQ ID NO: 21, such as at least 85%, 90%, 95% or even 100% identity to the mannanase of any of SEQ ID NO: 8, SEQ ID NO: 18, SEQ ID NO: 19, SEQ ID NO: 20 or SEQ ID NO: 21.
13) The detergent composition according to any of embodiments 10 to 12 further comprising a xanthan lyase having at least 80% identity to SEQ ID NO: 30 and a xanthan endoglucanase having at least 80% identity to SEQ ID NO: 31.
14) Use of the variant of any of embodiments 1 to 8, the enzyme composition according to embodiment 9 or the detergent composition of embodiment 10 to 12 in a cleaning process, such as laundry or hard surface cleaning such as dishwashing.
15) A method of cleaning an item, comprising exposing the item to a wash liquor comprising the variant of any of embodiments 1 to 8, the enzyme composition according to embodiment 9, or the detergent composition of embodiment 10 or 12, e.g. wherein the item is a textile or a hard surface.
16) A method for laundering a textile, comprising:
   a) Exposing the textile to a wash liquor comprising the variant of any of embodiments 1 to 8, the enzyme composition according to embodiment 9, or the detergent composition of embodiment 10 or 12;
   b) Completing at least one wash cycle; and optionally
   c) Rinsing the textile.
17) The detergent composition, use or method according to any of embodiments 10 to 14 comprising one or more enzymes selected from the group consisting of amylases, proteases, peroxidases, cellulases, betaglucanases, xyloglucanases, hemicellulases, xanthanases, xanthan lyases, lipases, acyl transferases, phospholipases, esterases, laccases, catalases, aryl esterases, amylases, alpha-amylases, glucoamylases, cutinases, pectinases, pectate lyases, keratinases, reductases, oxidases, phenoloxidases, lipoxygenases, ligninases, carrageenases, pullulanases, tannases, arabinosidases, hyaluronidases, chondroitinases, xylanases, pectin acetyl esterases, polygalacturonases, rhamnogalacturonases, other endo-beta-mannanases, exo-beta-mannanases, pectin methylesterases, cellobiohydrolases, transglutaminases, licheninases, laminarinases, and DNAses, or any combination thereof.
18) A polynucleotide encoding the variant of any of embodiments 1 to 8, a nucleic acid construct or an expression vector comprising the polynucleotide, or a recombinant host cell transformed with the polynucleotide.
19) A method for producing the variant of any of embodiments 1 to 8, comprising:
   a) Cultivating the recombinant host cell of embodiment 17 under conditions suitable for expression of the variant; and
   b) Recovering the variant.

### EXAMPLES

The present invention is further described by the following examples that should not be construed as limiting the scope of the invention.

### Strains

The DNA encoding the GH26 mannanase genes was isolated from Paenibacillus illinoisensis isolated from soil samples collected in Virginia, United States in 2014 and from Paenibacillus sp. isolated from a sand sample collected in The United States in 1991, and sequenced as described in WO 2019/068715.

### Materials

Chemicals used as buffers and substrates were commercial products of at least reagent grade.

### Model Detergent Systems

TABLE 1A
Model detergent 1 composition (liquid)

| Ingredient | | |
|---|---|---|
| (abbreviation) | Explanation | Wt % |
| LAS | (C10-C13)alkylbenzene-sulfonic acid | 12.0 |
| SLES | sodium lauryl ether sulfate | 4.1 |
| | Topped Palm Kernel Fatty Acid | 3.0 |
| AEO | alcohol ethoxylate | 12.0 |
| NaOH | Sodium hydroxide | 0.5 |
| Ethanol | Ethanol | 6.0 |
| MPG | monopropylene glycol | 2.0 |
| TEA | Triethanolamine | 2.0 |
| Sodium citrate | Sodium citrate dihydrate | 3.9 |
| DTMPA | diethylenetriaminepentakis(methylene)pentakis(phosphonic | 1.6 |
| | acid), heptasodium salt | |
| Phenoxyethanol | Phenoxyethanol | 0.5 |
| Ion exchanged water | Water | to 100 |

TABLE 1B
Model detergent 2 composition (liquid)

| Ingredient | Wt % |
|---|---|
| Llinear alkylbenzene sulfonic acid (LAS acid) | 22.0 |
| Alcohol ethoxylate with 7 mol EO (AEO-7) | 26.20 |
| Topped Palm Kernel Fatty Acid | 9.4 |
| Monoethanol amine (MEA) | 7.1 |
| Glycerol | 8.4 |
| Propylene glycol (MPG) | 15.7 |
| Diethylenetriaminepentakis(methylene)pentakis(phosphonic | 0.5 |
| acid), heptasodium salt (DTPMP Na7) | |
| Water (demineralized) | To 100 |
| pH target 7.5-8.0 | 8.0 |

### Swatches

The swatches include a combination of food and technical stains.

| Material | Source |
|---|---|
| DN-33 Fenugreek Seed | CFT |
| C-S-43 Guar Gum | CFT |
| C-S-73 Locust Bean Gum | CFT |

The above commercial test materials are available from Center for Testmaterials BV, Stoomloggerweg 11, 3133 KT Vlaardingen, the Netherlands.

### Wash Performance

### Terg-O-Tometer (TOM) Washing Trial

The terg-o-tometer is an industry standard. 1 L of wash solution is incubated in a water bath temperature-controlled environment. The solution is mixed for 5 min before adding 1 L to each of the beakers. The temperature in the beakers is measured to be 20.0° C. The washed and rinsed swatches are left to dry overnight in a drying cabinet and measured as indicated in table 2 below.

TABLE 2
Conditions for Terg-O-tometer Washing Trial

| Detergent dosage | Model detergent 1: 3.33 g/L or | |
|---|---|---|
| | Model detergent 2: 1.9 g/L | |
| Test solution volume | 1 | L |
| pH | unadjusted | |
| Wash time | 20 | minutes |
| Temperature | 20° | C. |
| Water hardness | 15° | dH |
| Ca²⁺:Mg²⁺:CO₃²⁻ ratio | 4:1:7.5 | |
| Mechanical action | 120 | rpm |

Wash performance is expressed as a delta remission value (ΔRem). After washing and rinsing the swatches are spread out flat and allowed to air dry at room temperature overnight. Light reflectance evaluations of the swatches are done using a Macbeth Color Eye 7000 reflectance spectrophotometer with large aperture. The measurements are made without UV in the incident light and remission at 460 nm is extracted. The dry swatches are measured with ColorEye 2. Measurement with small aperture through 3 layers (3 of the same type of swatch from the same beaker), 2 measurements on each swatch on the front side marked with beaker and swatch number. Remission values for individual swatches are calculated by subtracting the remission value of the control swatch from the remission value of the washed swatch. Calculating the enzyme effect is done by taking the measurements from washed swatches with enzymes and subtract with the measurements from washed without enzyme for each stain. The total enzyme performance is calculated as the average of individual ΔRem.

### Example 1: Reducing End Assay for Determination of Mannanase Activity

For estimating the mannose yield after substrate hydrolysis, a reducing end assay developed by Lever (1972), Anal. Biochem. 47: 273-279, is used. The assay is based on 4-hydroxybenzoic acid hydrazide, which under alkaline conditions reacts with the reducing ends of saccharides. The product is a strong yellow anion, which absorbs at 410 nm.

### Method

4-Hydroxybenzhydrazide (PAHBAH) (Sigma,H9882) is diluted in PAHBAH buffer to a concentration of 15 mg/ml. PAHBAH buffer contained: 50 g/L K—Na-tartrate (Merck, 1.08087) and 20 g/L sodium hydroxide(Sigma, S8045). This PAHBAH mix was made just before usage. 70 μl PAH BAH mix and MiliQ water are mixed in a 96 well PCR plate (Thermo Scientific). Samples from hydrolysis experiment were added. Samples and MiliQ always reached the total volume of 150 μl, but the dilution of the sample differed. The plate is sealed with Adhesive PCR Sealing Foil Sheets (Thermo Scientific). Plates are incubated at 95° C. for 10 min, cooled down and kept at 10° C. for 1 min in PTC-200 Thermal Cycler (MJ Research). 100 μl sample is transferred to a 96 well microtiter plate, flat bottomed (Nunc™) and color development measured at 405 nm on a SpectraMax 190 Absorbance Microplate Reader (Molecular Devices). Results are compared to mannose standards, that had undergone the same treatment and dilution as the samples to which they were compared.

### Example 2: Variant Generation by Site-Directed Mutagenesis

Site-directed variants of SEQ ID NO: 2 were generated by SOE (Splicing by Overlap Extension) simultaneously adding mutation(s), expression regulatory elements, and bacillus genome homology regions to the PCR product for site-directed integration. Bacillus subtilis was transformed with the PCR product; correct gene sequences were confirmed by NGS.

### Example 3: In-Detergent Stability Determination

Variants generated as described above were tested for in-detergent stability under the relevant conditions. In-detergent stability is performed by incubating the variants in detergent comprising 490 ppm protease (SEQ ID NO:17) and subsequently calculate the half-life of the variants.

The following procedure was applied (conditions indicated in Table 3):
1) Dissolve mannanase variant in 0.01% Triton x-100 buffer (Triton X-100 Sigma-Aldrich Product No.: 93426, CAS No. 9036-19-5)
2) Add the mannanase variant to the model detergent comprising protease to obtain the desired final mannanase concentration and stir for 30 minutes
3) Incubate the enzyme/detergent solution at the desired temperature
4) Samples of the enzyme/detergent solution are analyzed for residual mannanase activity after 0 hours, 24 hours, 72 hours and 144 hours and the half-life are calculated.

The residual mannanase activity is measured by using Mannazyme Tablets from Megazyme which is a substrate for endo-1,4-β-mannanase. The Mannazyme Tablet substrate is dissolved in 100 mM MOPS (3-(N-morpholino)propanesulfonic acid, CAS No: 1132-61-2) and incubated with enzyme/detergent solution at room temperature for 60 minutes, shaking at 800 rpm. Spin down at 2000 rpm for 2 minutes to allow insoluble substrate to settle. and mannanase activity is measured by reading the optical density of supernatant at 590 nm.

The half-life (T_{1/2}), i.e., the time where 50% of the mannanase activity remains, is calculated. Half-life for SEQ ID NO:2, SEQ ID NO:3 and SEQ ID NO:4 was obtained in the above disclosed way, results are provided in Table 3. It is clear that the mannanase variants having SEQ ID NO:3 and SEQ ID NO:4 are significantly improved in terms of in-detergent stability compared to the mannanase variant having SEQ ID NO:2.

TABLE 3
Half-life of variants of the invention

| | Incubation | Mannanase | | Half |
|---|---|---|---|---|
| SEQ ID | temperature | concentration in | | life |
| NO | (° C.) | detergent (ppm) | Detergent | (hours) |
| 2 | 42 | 38.5 | Model Detergent 1 | 48 |
| 3 | 42 | 38.5 | Model Detergent 1 | 471 |
| 4 | 42 | 38.5 | Model Detergent 1 | 482 |

### Example 4: Residual Activity

600 ppm protease (SEQ ID NO: 17) and mannanase was added to Model Detergent 1 and Model Detergent 2. Samples for stability test were stored at 37° C. for 4 weeks, closed lids, whereas reference samples were stored at minus 18° C. for 4 weeks. The residual activity was calculated as:

Activity (incubated sample)/Activity (reference sample)*100%

For measurement of activity the the principle of the reducing end assay described in Example 1 is applied with the following particularities: 5 gram detergent sample is dissolved in 400 mL buffer (0.1 M NaH2PO4, 2H2O and 0.05% (w/v) TWEEN® 20, pH 7.3) and further diluted in 0.05M HEPES buffer (pH 8.0) to be within a relevant concentration range.

The residual activity was measured with the results listed in Table 4.

TABLE 4
Residual activity after storage

| SEQ ID | Mannanase concentration | | Residual |
|---|---|---|---|
| NO | in detergent (ppm) | Detergent | activity |
| 2 | 20 | Model Detergent 1 | 3% |
| 3 | 20 | Model Detergent 1 | 75% |
| 4 | 20 | Model Detergent 1 | 67% |
| 5 | 20 | Model Detergent 1 | 31% |
| 6 | 20 | Model Detergent 1 | 71% |
| 7 | 20 | Model Detergent 1 | 60% |
| 2 | 20 | Model Detergent 2 | 5% |
| 3 | 20 | Model Detergent 2 | 37% |
| 4 | 20 | Model Detergent 2 | 53% |
| 5 | 20 | Model Detergent 2 | 61% |
| 6 | 20 | Model Detergent 2 | 41% |
| 7 | 20 | Model Detergent 2 | 59% |

It is clear from the data in Table 4 that the storage stability in detergent is much improved for the mannanase variants having SEQ ID NO: 3 to 7 compared to the storage stability in detergent of the mannanase known from the prior art (SEQ ID NO: 2).

### Example 5: Residual Wash Performance (RWP) of Mannanase Variants Using Terg-O-Tometer

Variants generated as described above were tested for RWP under the relevant conditions. RWP is performed by incubating the variants in detergent comprising 490 ppm protease (SEQ ID NO:17) and subsequently evaluate the RWP using the TOM washing trial method as described above. RWP was calculated as

Wash Performance (incubated sample)/Wash Performance (reference sample)*100%

where the reference sample is prepared the same way as the incubated sample but stored at minus 18° C. for 4 weeks. RWP reflects the average of individual Delta REM for the three types of swatches described above.

TABLE 5
RWP after storage

| | | Mannanase | | |
|---|---|---|---|---|
| | | concentration | | |
| SEQ ID | Incubation | in wash | | |
| NO | condition | liquor (ppm) | Detergent | RWP |
| 2 | 37° C./4 weeks | 0.02 | Model Detergent 1 | 8% |
| 3 | 37° C./4 weeks | 0.02 | Model Detergent 1 | 70% |
| 4 | 37° C./4 weeks | 0.02 | Model Detergent 1 | 83% |
| 5 | 37° C./4 weeks | 0.02 | Model Detergent 1 | 59% |
| 6 | 37° C./4 weeks | 0.02 | Model Detergent 1 | 85% |
| 7 | 37° C./4 weeks | 0.02 | Model Detergent 1 | 85% |

It is clear from the data in Table 5 that the residual wash performance upon storage in detergent of the mannanase variants having SEQ ID NO: 3 to 7 is much improved over the mannanase known from the prior art (SEQ ID NO: 2).

### Example 6: Half-Life Improvement Factor for Variants of SEQ ID NO: 3

Stability test was performed by incubating the variants in detergent for different length of time and temperature and comparing the activity against control having SEQ ID NO: 3 which was incubated at 4° C. for the same duration.

For the variants in Table 6, the stress conditions consisted of incubating the variants in Model Detergent 1 and the protease having SEQ ID NO:32 (1471 μM) at 42° C. for 24 hours.

The residual activity was measured using the Mannanase enzyme assay using insolubleAzo-carob-galactomannan substrate from Megazyme. Substrate was incubated with enzyme at 30′C for 30 min, shaking at 800 rpm. After this, the reaction mixture was kept static for 10 min to allow insoluble substrate to settle. Enzyme activity was measured by reading the optical density of supernatant at 590 nm. The residual activity (RA) was calculated by taking the ratio of Stress response to Un-stress response, which is then used to calculate half-life. The half-life was calculated according to the following equation, where RA=residual activity, t=incubation time in hours, and half-life is defined in hours:

$$\frac{Ln ( 0. 5 )}{( \frac{Ln ( RA )}{t} )} \vert$$

Half-life Improvement Factor (HIF) is calculated by taking the Half-life ratio of the sample variant to the mannanase having SEQ ID NO: 3 which are also grown on the replicate microtiter plate.

TABLE 6
Half-life improvement factor for variants of SEQ ID NO: 3

| Substitutions compared to SEQ ID NO: 3 | HIF in Model Detergent 1 |
|---|---|
| — | 1.00 |
| R164P, I165E, P166D | 0.77 |
| R164W, I165P | 0.68 |
| R164I, I165P | 0.99 |
| R164S, I165G (SEQ ID NO: 22) | 1.00 |
| R164P, I165A (SEQ ID NO: 23) | 1.10 |
| R164A, I165A (SEQ ID NO: 24) | 1.33 |
| R164D, I165A | 1.32 |
| A163S, R164L, I165T | 1.08 |
| A163P, R164L, I165T | 1.11 |
| V161A, R164K, I165A (SEQ ID NO: 25) | 0.98 |
| R164P, I165P (SEQ ID NO: 26) | 1.17 |
| R164P | 0.90 |
| I165P | 0.00 |
| R164P, I165E | 0.99 |
| R164P, I165G | 0.98 |
| R164P, I165T (SEQ ID NO: 27) | 1.45 |
| R164W, I165E | 0.91 |
| R164W, I165G | 0.96 |
| R164W, I165A | 1.05 |
| R164W, I165T | 0.97 |
| R164I, I165E | 1.66 |
| R164I, I165G | 1.03 |
| R164I, I165A | 1.22 |
| R164I, I165T | 1.12 |
| R164A, I165P | 0.92 |
| R164A, I165E (SEQ ID NO: 28) | 1.03 |
| R164A, I165G | 0.97 |
| R164A, I165T | 1.35 |
| R164P, I165P, P166D | 0.93 |
| R164A, I165P, P166D | 0.78 |
| R164I, I165P, P166D | 0.00 |
| R164W, I165P, P166D | 0.56 |
| A163P, R164I, I165P | 1.44 |
| A163P, R164P, I165T | 1.31 |
| V161A, R164P, I165P | 1.09 |
| V161A, R164P, I165A | 1.14 |
| V161A, R164A, I165P (SEQ ID NO: 29) | 0.71 |
| V161A, R164A, I165A | 1.01 |
| A163P, R164I, I165P, P166D | 0.69 |
| A163P, R164P, I165T, P166D | 0.98 |
| V161A, R164P, I165P, P166D | 0.44 |
| V161A, R164P, I165A, P166D | 1.01 |
| V161A, R164A, I165P, P166D | 0.92 |
| V161A, R164A, I165A, P166D | 0.52 |

Examples 3 and 4 show that the variant having SEQ ID NO: 3 is improved over the mannanase known from the prior art (SEQ ID NO: 2). It is clear from the data in Table 6 that the stability of the variants of SEQ ID NO: 3 in the presence of protease is further improved.

### Example 7: Synergetic Action of Mannanase with Other Enzymes on Stain Removal

The synergetic action of mannanase together with 4 different laundry enzymes is tested on 9 different stains in a wash trial assay.

### Materials

Chemicals used as buffers and substrates were commercial products of at least reagent grade.

### Model Detergent System

Model detergent 1 as disclosed in Table 1A was used.

### Enzymes

TABLE 7
Enzymes tested

| Enzyme name | Functional description | Dosage |
|---|---|---|
| SEQ ID NO: 3 | GH26 Mannanase | 0.01 ppm |
| *Caledonia 100L | Xanthanase | 0.45 Wt % |
| *Lipex Evity 200L | Lipase | 0.20 Wt % |
| *Amplify Prime 100L | Amylase | 0.20 Wt % |
| *Liquananse Evity 3.5L | Protease | 0.80 Wt % |

*The above commercial enzymes are from Novozymes A/S
Caledonia ® 100L comprises the xanthan lyase and the xanthan endoglucanase having SEQ ID NO: 31 and SEQ ID NO: 32, respectively.

### Stains

The stains include a combination of food and technical stains.

TABLE 8
Stain types

| Code | Soil description | Sensitivity | Source |
|---|---|---|---|
| WE5GMWKC | Grass/Mud | Protease | Equest |
| E5DASBWKC | Sheep's Blood Double | Protease | Equest |
| C-S-406 | Balsamico salad | Amylase | CFT |
| | dressing | | |
| WE5BBWKC | Cooked Beef fat | Lipase/Surfactant | Equest |
| 132KC | Cooked butter | Lipase/Surfactant | Equest |
| C-S-44 | Chocolate drink, pure | Mannanase/protease | CFT |
| C-2-68 | Chocolate ice cream, | Mannanase/ | CFT |
| | aged, on woven cotton | Xanthanase | |
| C-S-02 | Cocoa, aged, on | Protease/Mannanase/ | CFT |
| | woven cotton | Xanthanase | |
| C-S-05S | Mayonnaise w. Carbon | Mannanase/Lipase | CFT |
| | black | | |

CFT: Center for Testmaterials BV, Stoomloggerweg 11, 3133 KT Vlaardingen, the Netherlands.
Equest: Warwick Equest, The United Kingdom

### Prewashed White Ballast

Ballast consisting of 50%:50% (polyester:cotton) fabric is prewashed with amylase and cellulase to reduce/remove starch, carboxymethyl cellulose (CMC) and other textile additives. 3 kg of textile ballast is washed three times in 78.6 g of W-ECE-2 detergent (wfk Testgewebe GmbH, Germany) using water with 15°dH water hardness [Ca²⁺:Mg²⁺:CO₃²⁻ ratio, 4:1:7.5] and containing the following enzymes for each of the three prewash steps.

TABLE 9
Prewashing steps

| | Dosage (g/wash) | | |
|---|---|---|---|
| Enzyme | Prewash 1 | Prewash 2 | Prewash 3 |
| Stainzyme 12L (Amylase) | 3 | 3 | 0 |
| Celluclean 4500T (Cellulase) | 3.75 | 0 | 0 |

Prewashing is carried out in a Miele Softtronic W1935 WTL machine at 40° C. using a standard washing program with 13-15 liters of water for 3 kg of textile. After the third prewash step, a rinse is carried out in deionized water and textile fabrics are then line dried and cut into 5 cm×5 cm pieces.

### Wash Performance

Enzyme synergy test was tested in a Terg-O-tometer (TOM) washing trial under the conditions described above to determine wash performance (stain removal efficiency). General wash conditions are given in Table 2, enzymes were added at the specified dosages (Table 7) for washes with enzymes. Two stains of each stain type (Table 8) were included in each beaker together with prewashed ballast so total weight is approximately 30 g/beaker. The washed and rinsed stains were left to dry overnight in a drying cabinet at room temperature and stain removal is assessed using a Datacolor 800V spectrophotometer (Datacolor, Lawrenceville, NJ, USA). Light reflectance evaluation of the stains was performed under the CIE Standard Illuminant D65 and the CIE 1964 10-degree Standard Observer. The measurements were made without UV in the incident light and remission values at 460 nm were recorded for each stain washed with and without enzymes. Wash performance is expressed as the average remission value (REM) at 460 nm of the two stains of each stain type.

### Results

This example shows that the total stain removal efficiency (Table 10, “Sum of all 9 stains”) is higher when GH26 mannanase is used together with only the xanthanase, (Table 10, “M/X”) compared to the controls (“B”, “M” and “X”) in the combination. An increased total stain removal efficiency is also observed if GH26 mannanase is used in a multienzyme combination together with amylase/protease (Table 10, “A/P”), amylase/protease/lipase (Table 10, “A/P/L”) and amylase/protease/lipase/xanthanase (Table 10, “A/P/L/X”) when compared to their respective controls. The observed synergetic action of GH26 mannanase with laundry enzymes on stain removal is dependent on the stain type and thus the soil substrate. For example, no synergy of the GH26 mannanase together with tested enzymes is detected on “Grass/Mud”, “Cooked beef fat” and “Cooked butter” while it is evident that certain stains are more effective in demonstrating synergetic action of GH26 mannanase together with particular enzymes on stain removal.

TABLE 10
Remission values at 460 nm (stain removal efficiency)

| | | | | | | C-S-68 | | WE5DASBW | | |
|---|---|---|---|---|---|---|---|---|---|---|
| | C-S-05S | | | | | Chocolate | C-S-406 | KC | | |
| *Sum | Mayonnaise | C-S-02 | C-S-44 | 132KC | WE5BBWKC | ice | Balsamico | Sheep's | | |
| of all 9 | w. carbon | Cocoa, | Chocolate | Cooked | Cooked | cream, | salad | Blood | WE5GMWKC | |
| stains | black | aged | drink, pure | butter | beef fat | aged | dressing | Double | Grass/Mud | Stains |
| 342.4 | 26.5 | 32.3 | 35.5 | 61.5 | 48.5 | 41.1 | 24.1 | 39.3 | 33.7 | B |
| 346.5 | 26.0 | 33.7 | 37.4 | 62.7 | 48.1 | 39.4 | 25.5 | 39.9 | 33.7 | M |
| 369.4 | 30.5 | 35.1 | 36.4 | 62.8 | 49.9 | 43.1 | 28.0 | 47.0 | 36.6 | P |
| 368.0 | 31.9 | 36.2 | 37.0 | 63.2 | 46.9 | 47.6 | 27.7 | 43.6 | 34.0 | M/P |
| 352.9 | 27.9 | 33.9 | 38.3 | 62.5 | 49.3 | 40.5 | 25.2 | 40.2 | 35.1 | A |
| 350.0 | 27.1 | 32.2 | 37.0 | 62.6 | 49.3 | 42.9 | 26.1 | 38.4 | 34.4 | M/A |
| 363.0 | 27.0 | 37.5 | 37.8 | 66.6 | 51.9 | 43.2 | 23.8 | 40.5 | 34.8 | L |
| 363.6 | 28.1 | 34.8 | 37.3 | 66.8 | 49.1 | 45.7 | 25.9 | 41.8 | 34.0 | M/L |
| 347.2 | 25.9 | 32.6 | 37.7 | 62.6 | 46.9 | 40.3 | 25.2 | 38.5 | 37.4 | X |
| 361.6 | 30.1 | 36.2 | 39.5 | 62.9 | 48.0 | 45.8 | 24.4 | 40.8 | 33.9 | M/X |
| 368.4 | 29.7 | 36.1 | 38.3 | 60.9 | 47.3 | 42.9 | 33.3 | 43.9 | 36.1 | A/P |
| 381.7 | 31.7 | 38.3 | 36.2 | 63.6 | 47.2 | 47.5 | 37.5 | 44.4 | 35.4 | M/A/P |
| 379.5 | 27.2 | 39.6 | 37.4 | 65.4 | 47.1 | 48.6 | 34.4 | 43.2 | 36.6 | A/P/L |
| 398.1 | 32.6 | 40.5 | 39.1 | 67.0 | 50.7 | 50.9 | 35.0 | 46.8 | 35.5 | M/A/P/ |
| | | | | | | | | | | L |
| 387.5 | 30.3 | 37.8 | 40.6 | 65.9 | 47.6 | 49.4 | 33.1 | 48.4 | 34.4 | A/P/L/ |
| | | | | | | | | | | X |
| 405.7 | 35.7 | 44.3 | 41.5 | 66.8 | 47.7 | 50.7 | 40.1 | 44.5 | 34.6 | M/A/P/ |
| | | | | | | | | | | L/X |

Values represent the average of 2 stains of each type.
*Sum is the total sum of the average remission values of each stain type
B: Blank-Detergent alone
A: Amylase (Amplify Prime ® 100 L) M: Mannanase SEQ ID NO: 3
P: Protease (Liquananse Evity ® 3.5 L) L: Lipase (Lipex Evity ® 200 L) X: Xanthanase (Caledonia ® 100 L)

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1-21. (canceled)

22. A variant of the polypeptide of SEQ ID NO: 2 wherein
the amino acids at position 490 and 491 are deleted,
the variant has at least 60% sequence identity to the polypeptide of SEQ ID NO: 3 and
the variant has mannanase activity.

23. The variant according to claim 22, wherein the variant further comprises an extension of one or more amino acids at the N-terminal and/or C-terminal ends or a truncation of one or more amino acids at the N-terminal and/or C-terminal ends and wherein an insertion at the C-terminal is not *490W and *491R.

24. The variant according to claim 22, further comprising a deletion of at least 11 amino acids from the N-terminal end.

25. The variant according to claim 22, further comprising one or more modifications selected from the group consisting of I30L, D48P, Y155H, T167P, Q215E, H276C, R280K, F286C, G366N, and D486E.

26. The variant according to claim 25, further comprising one or more modifications selected from the group consisting of G20P, A101L, A111P, A118E, S137A, Q143R, R160L, E162D, M171I, N176S, P182R, Q183E, V244A, H324K, and D385H.

27. The variant according to claim 22, wherein the variant has at least 65% sequence identity to the polypeptide of SEQ ID NO: 3, the polypeptide of SEQ ID NO: 4, the polypeptide of SEQ ID NO: 5, the polypeptide of SEQ ID NO:6, the polypeptide of SEQ ID NO: 7, the polypeptide of SEQ ID NO: 22, the polypeptide of SEQ ID NO: 23, the polypeptide of SEQ ID NO: 24, the polypeptide of SEQ ID NO: 25, the polypeptide of SEQ ID NO: 26, the polypeptide of SEQ ID NO: 27, the polypeptide of SEQ ID NO: 28, or the polypeptide of SEQ ID NO: 29, wherein the variant has mannanase activity.

28. The variant of claim 22, wherein the variant has at least 70% sequence identity to the polypeptide of SEQ ID NO: 3, the polypeptide of SEQ ID NO: 4, the polypeptide of SEQ ID NO: 5, the polypeptide of SEQ ID NO:6, the polypeptide of SEQ ID NO: 7, the polypeptide of SEQ ID NO: 22, the polypeptide of SEQ ID NO: 23, the polypeptide of SEQ ID NO: 24, the polypeptide of SEQ ID NO: 25, the polypeptide of SEQ ID NO: 26, the polypeptide of SEQ ID NO: 27, the polypeptide of SEQ ID NO: 28, or the polypeptide of SEQ ID NO: 29.

29. The variant of claim 22, wherein the variant has at least 80% sequence identity to the polypeptide of SEQ ID NO: 3, the polypeptide of SEQ ID NO: 4, the polypeptide of SEQ ID NO: 5, the polypeptide of SEQ ID NO:6, the polypeptide of SEQ ID NO: 7, the polypeptide of SEQ ID NO: 22, the polypeptide of SEQ ID NO: 23, the polypeptide of SEQ ID NO: 24, the polypeptide of SEQ ID NO: 25, the polypeptide of SEQ ID NO: 26, the polypeptide of SEQ ID NO: 27, the polypeptide of SEQ ID NO: 28, or the polypeptide of SEQ ID NO: 29.

30. The variant of claim 22, wherein the variant has at least 90% sequence identity to the polypeptide of SEQ ID NO: 3, the polypeptide of SEQ ID NO: 4, the polypeptide of SEQ ID NO: 5, the polypeptide of SEQ ID NO:6, the polypeptide of SEQ ID NO: 7, the polypeptide of SEQ ID NO: 22, the polypeptide of SEQ ID NO: 23, the polypeptide of SEQ ID NO: 24, the polypeptide of SEQ ID NO: 25, the polypeptide of SEQ ID NO: 26, the polypeptide of SEQ ID NO: 27, the polypeptide of SEQ ID NO: 28, or the polypeptide of SEQ ID NO: 29.

31. An enzyme composition comprising the variant of claim 22 and a GH5 mannanase.

32. A detergent composition comprising the variant of claim 22 and at least one detergent adjunct ingredient.

33. The detergent composition according to claim 32, further comprising a GH5 mannanase.

34. The detergent composition according to claim 33, wherein the ratio between the GH5 mannanase and the variant is in the range from 1:3 to 3:1 based on weight of enzyme protein.

35. The detergent composition according to claim 32, further comprising a xanthan lyase having at least 80% identity to SEQ ID NO: 30 and a xanthan endoglucanase having at least 80% identity to SEQ ID NO: 31.

36. A method of cleaning an item, comprising exposing the item to a wash liquor comprising the variant of claim 22.

37. The method of claim 36, wherein the item is a textile or a hard surface.

38. A polynucleotide encoding the variant of claim 22.

39. A nucleic acid construct or an expression vector comprising the polynucleotide of claim 38.

40. A recombinant host cell transformed with the polynucleotide of claim 38.

41. A method for producing a mannanase variant, comprising:
a) cultivating the recombinant host cell of claim 40 under conditions suitable for expression of the mannanase variant; and
b) recovering the mannanase variant.
